(19) United States
(12) Patent Application Publication
(10) Pub. No.: US 2025/0207102 A1
(43) Pub. Date: Jun. 26, 2025

(54) METHODS FOR CO-CULTURING GENOTOXIC BACTERIA AND ORGANOIDS

(71) Applicant: HUB Organoids IP B.V., Utrecht (NL)

(72) Inventors: Carla Sofia Leiria Verissimo, Utrecht (NL); Roshni Rajshekhar Nair, Utrecht (NL); Sylvia Fernandez Boj, Utrecht (NL); Eider Valle Encinas, Utrecht (NL)

(73) Assignee: HUB Organoids IP B.V., Utrecht (NL)

(21) Appl. No.: 18/850,773

(22) PCT Filed: Mar. 27, 2023
(86) PCT No.: PCT/EP2023/057803

§ 371 Date: Sep. 25, 2024

(30) Foreign Application Priority Data
Mar. 25, 2022 (GB) 2204266.7

(51) Int. Cl. C12N 5/071; C12N 1/20

(52) U.S. Cl. CPC C12N 5/0679; C12N 1/20; C12N 2503/04; C12N 2533/90

(57) ABSTRACT

The invention relates to methods for co-culturing bacteria and organoids, particularly genotoxic bacteria and organoids. It also relates to methods of producing an organoid with a mutational signature. It also relates to uses of the co-cultures and organoids, for example in methods for testing the effect of a candidate compound, modelling host-pathogen interactions, or identifying compounds suitable for treating bacterial infection and/or cancer.

### FIELD

The invention is in the field of co-culturing genotoxic bacteria and organoids and in particular the production of organoids with cancer mutational signatures by co-culturing genotoxic bacteria and organoids.

### BACKGROUND

Organoids are a promising tool to study human physiology in vitro. Organoids are self-organized epithelial cell structures with physiological features that resemble their in vivo organization. They have been extensively used to model aspects of cancer initiation and progression. Stem-cell-derived organoids therefore provide sophisticated models for studying human development and disease.

The intestinal microbiome has long been thought to be involved in colorectal cancer (CRC) tumorigenesis. Various bacterial species have been reported to be enriched in stool and biopsy samples from patients with CRC, including genotoxic strains of E. coli. The genome of these genotoxic bacteria harbours a polyketide non-ribosomal peptide synthase operon (pks) that is responsible for the production of the genotoxin colibactin. Colibactin induces DNA double-strand breaking, chromosome aberrations, and cell cycle arrest in the G2/M phase [1].

Microinjection of colibactin-producing pks+E. coli into the lumen of human intestinal organoids resulted in the appearance of two co-occurring mutational signatures identified in a subset of CRC patients, demonstrating that pks+E. coli plays a causative role in CRC development [2]. The scalability of the microinjection model is, however, limited and represents a bottleneck in the screening of preventative therapies for these patients. Therefore, there is a need in the art for improved methods for co-culturing bacteria and organoids that can produce organoids with a mutational signature, such as a cancer mutational signature.

### SUMMARY OF THE INVENTION

The invention provides a method for co-culturing genotoxic bacteria and organoids, wherein the method comprises at least two phases selected from a first phase comprising culturing organoids or organoid fragments with genotoxic bacteria; a second phase comprising culturing the organoids or organoids fragments with genotoxic bacteria, wherein the bacterial growth is restricted with antibiotics, a third phase comprising killing the genotoxic bacteria and enabling the further growth of the organoids or organoid fragments.

The invention also provides a method of producing an organoid with a mutational signature, wherein the method comprises at least two phases selected from: a first phase comprising culturing organoids or organoid fragments with genotoxic bacteria; a second phase comprising culturing the organoids or organoids fragments with genotoxic bacteria, wherein the bacterial growth is restricted with antibiotics, a third phase comprising killing the genotoxic bacteria and enabling the further growth of the organoids or organoid fragments, wherein an organoid or an organoid fragment that has a mutational signature is produced.

The invention also provides a method for testing the effect of a candidate compound, wherein the method comprises: exposing the co-culture of genotoxic bacteria and organoids from the methods of the invention to one of a library of candidate compounds; evaluating the organoids in the co-culture for any effects, identifying the candidate molecule that causes said effects as a potential drug.

The invention also provides organoids with a mutational signature produced by the methods of the invention and uses of these organoids in modelling host-pathogen interaction and/or for identifying compounds suitable for treating bacterial infection and/or cancer.

### BRIEF DESCRIPTION OF THE DRAWINGS

FIG. 1—(A) A schematic of a method for co-culturing genotoxic bacteria and organoids consisting of three phases: the first phase (no antibiotic), the second phase (low antibiotic concentration) and the third phase (high antibiotic concentration); (B) Immunofluorescence of organoid fragments after 4 h of co-culture with pks E. coli MOI 100 in the first phase; stained using DAPI (blue, which stains nuclei), Phalloidin (red, which stains actin filaments) and bacteria (green). The DAPI staining shows the presence of the nuclei in the organoids. The phalloidin dye stains the apical side of the organoid fragments. FIG. 1B shows that the apical surface of the organoid fragments is in contact with the bacteria strained in green. (C) Distribution of the organoid single cells derived from the genotoxic bacteria and organoid co-culture as a function of yH2AX levels and at different time points, as obtained by flow cytometry; (D) Representative image of organoids at the end of the recovery period. MOI 0=[Bact]1, MOI 10=[Bact]2, MOI 50=[Bact]3, MOI 100=[Bact]4.

FIG. 2—(A) A schematic of a method for co-culturing genotoxic bacteria and organoids that consisted of two phases—the first phase (co-culture I) and the third phase (recovery). 0% MG=zero Matrigel in the culture; (B) Organoids after the recovery period in the absence of bacteria (MOI 0), in the presence of colibactin producing bacteria (WT MOI 500) and in the presence of colibactin-deficient (ΔCIbP MOI 500), stained using DAPI (blue, recognizes cell nuclei) and a yH2AX stain (top row, red) and yH2AX stain only (bottom row, red) and imaged using immunofluorescence. The yH2AX stain shows where the DNA damage occurred in the organoids. The bottom panel shows the presence of the DNA damage in the organoids and the top panel shows the location of this DNA damage relative to the nuclei of the organoids; (C) Example immunofluorescence measurement of yH2AX and DAPI signal in the nuclear region in order to obtain average yH2AX/DAPI nuclear intensity; (D) Average yH2AX/DAPI nuclear intensity for organoids in the absence of bacteria (MOI 0), in the presence of colibactin producing bacteria (WT MOI 500) and in the presence of colibactin-deficient (ΔClbP MOI 500) bacteria as measured by immunofluorescence after the recovery period.

FIG. 3—(A) A schematic of a method for co-culturing genotoxic bacteria and organoids consisting of two phases: the first phase (co-culture I), which is in suspension with no antibiotics and the second phase (co-culture II), which is on 70% Matrigel (MG) with a low antibiotic concentration (50 μg/ml of gentamicin); (B) Organoid fragments survival after 3 h of co-culture with pks+E. coli (WT and ΔClbP) in suspension (the first phase) this the co-culture (I) from FIG. 3A; (C) Organoids after the recovery period in the absence of bacteria (MOI 0), in the presence of colibactin producing bacteria (WT MOI 300) and in the presence of colibactin-deficient (ΔClbP MOI 300), stained using DAPI (recognizes cell nuclei, blue) and a yH2AX stain (top row, red) and yH2AX stain only (bottom row, red) and imaged using immunofluorescence (IF). The yH2AX stain shows where the DNA damage occurred in the organoids. The right hand panel shows the presence of the DNA damage in the organoids and the left hand panel shows the location of this DNA damage relative to the nuclei of the organoids; (D) yH2AX/DAPI nuclear intensity after treatment with colibactin producing bacteria (WT MOI 50, WT MOI 100, WT MOI 200 and WT MOI 300) or after treatment with colibactin-deficient (ΔClbP MOI 50, MOI 100, MOI 200 and MOI 300).

### Methods for Co-Culturing Bacteria and Organoids

The invention provides methods of co-culturing bacteria and organoids that involve at least two phases selected from: a first phase comprising culturing organoids or organoid fragments with genotoxic bacteria; a second phase comprising culturing the organoids or organoid fragments with genotoxic bacteria, wherein the bacterial growth is restricted with antibiotics, and a third phase comprising killing the genotoxic bacteria and enabling the further growth of the organoids or organoid fragments. In some embodiments, the method produces an organoid or an organoid fragment with a mutational signature. Each phase can be conducted with organoids, organoid fragments or a combination of organoids and organoid fragments. During any phase, particularly in the second or third phase, the organoid fragments can naturally form one or more organoids. The organoids or organoid fragments produced by these methods may be useful as a screening platform for identifying new drugs or testing drug candidates, or as a model for identifying new biomarkers, particularly in the context of cancer. In addition, the methods of co-culturing bacteria and organoids can be used to test the effect of a candidate compound.

The first phase of the co-culture comprises culturing the organoids or organoid fragments with genotoxic bacteria. In this first phase the growth of the bacteria in the medium is typically unrestricted. Without wishing to be bound by any particular theory, this unrestricted bacterial growth results in “acute damage” to the DNA in the organoids or organoid fragments.

In the second phase of the co-culture, the growth of the genotoxic bacteria in the co-culture is restricted using an antibiotic. Accordingly, the quantity of bacteria in the second phase of the co-culture is reduced compared to the first phase. In the second phase the organoids or organoid fragments are subjected to a lower rate of DNA damage, referred to herein “sustained damage”.

In the third phase the bacteria in the co-culture are killed, for example using a high concentration of an antibiotic, and the organoids or organoid fragments are further cultured. This phase allows the recovery of the organoids or organoid fragments in the absence of genotoxic bacteria.

In some embodiments, the invention provides a method of co-culturing bacteria and organoids that involves the first and second phases only. In this embodiment the method for co-culturing genotoxic bacteria and organoids comprises (a) culturing organoids or organoid fragments with genotoxic bacteria (the first phase) and (b) culturing the organoids or organoid fragments with genotoxic bacteria, wherein the bacterial growth is restricted with antibiotics (the second phase). Without wishing to be bound by any particular theory, the combination of an acute phase of DNA damage followed by a sustained period of DNA damage produces a cancer mutational signature. The acute damage phase introduces large number of mutations into the organoids, but cannot be continued for a long period of time otherwise the organoids will die. By co-culturing the organoids in two stages, a short phase of acute DNA damage and a longer phase of sustained DNA damage, it is believed that the method can produce organoids with a cancer mutational signature without killing the organoids. Exposing the organoids to the genotoxic compound for a longer period of time is thought to be useful in ensuring that the organoids produced have a cancer mutational signature, for example a cancer mutational signature that faithfully represents the mutational signature that may occur in vivo.

In some embodiments, the invention provides a method of co-culturing bacteria and organoids that involves the first and the third phases only (known as a rapid assay). In some embodiments, the method for co-culturing genotoxic bacteria and organoids comprises (a) culturing organoids or organoid fragments with genotoxic bacteria (the first phase) and (b) killing the genotoxic bacteria and enabling the further growth of the organoids (the third phase). The above method is particularly useful as a screening platform for drugs, such as cancer drugs, because the rapid speed is suitable for medium to high throughput screening methods.

In some embodiments, the invention provides a method of co-culturing bacteria and organoids that involves the second and the third phase only. In some embodiments, the method for co-culturing genotoxic bacteria and organoids comprises (a) culturing the organoids or organoid fragments with genotoxic bacteria, wherein the bacterial growth is restricted with antibiotics (the second phase), and (b) killing the genotoxic bacteria and enabling the further growth of the organoids (the third phase). Without wishing to be bound by any particular theory, it is believed that a cancer mutational signature can be obtained after a prolonged period of low-level DNA damage. In patients the concentration of genotoxic compounds produced by bacteria in their microbiome is low. Therefore, the cells in a patient, such as the cells in the gastrointestinal tract or the lungs, will be exposed to low concentrations of genotoxic compounds over a long period of time. This version of the method aims to recreate this longer-term low-level exposure to genotoxic compounds that can result in certain types of cancer, such as colorectal cancer. This method is particularly useful for searching for new biomarkers, e.g. cancer biomarkers.

In some embodiments, the invention provides a method of co-culturing bacteria and organoids that involves the first, the second and the third phase. In some embodiments, the method for co-culturing genotoxic bacteria and organoids comprises (a) culturing organoids or organoid fragments with genotoxic bacteria (the first phase); (b) culturing the organoids or organoid fragments with genotoxic bacteria, wherein the bacterial growth is restricted with antibiotics (the second phase) and (c) killing the genotoxic bacteria and enabling the further growth of the organoids (the third phase). Without wishing to be bound by any particular theory, it is believed that the combination of an acute phase of DNA damage, followed by a phase of sustained DNA damage and then a recovery phase increases the yield of organoids with a mutational signature. The recovery phase ensures that the organoids can recover from the DNA damage that has been caused and repair any of the DNA breaks that have occurred. During this process of repair and recovery the organoids produce a mutational signature that can be similar to the mutational signatures observed in cancer patients. This method is particularly useful for searching for new biomarkers, e.g. cancer biomarkers.

In some embodiments, the phases in the method are repeated at least twice, at least three times, at least four times or at least five times. In preferred embodiments, the phases in the method are repeated at least five times. Repetition is particularly useful in the long-term exposure model (or any method which includes the second phase of “sustained damage”) because the aim of the long-term exposure model is to faithfully replicate DNA damage that may occur in vivo. Without wishing to be bound by any particular theory, patients who develop cancer are usually exposed to genotoxic compounds over a period of time, therefore repeating the phases allows the system to produce organoids that have a mutational signature which is similar to the mutational signature of a cancer, such as colorectal cancer. For example, Pleguezuelos-Manzano, et al. demonstrated that repeated exposure of human intestinal organoids to genotoxic E. coli by repeated luminal injection over five months resulted in mutational signature that was detected in a subset of cancer genomes [2]. Furthermore, patients are usually exposed over long periods of time to the genotoxic compounds that are produced by their microbiome. Therefore, organoids that are repetitively exposed to genotoxic bacteria are more likely to result in a mutational signature that is found in cancer patients.

The first phase, the second phase and/or the third phase can comprise culturing in the presence of an extracellular matrix, such as Matrigel or Basement Membrane Extract. In some embodiments, the second and/or the third phases comprise culturing in the presence of an extracellular matrix, such as Matrigel or Basement Membrane Extract.

The first phase, the second phase and/or the third phase can be conducted in suspension culture. This enables the organoids that are being cultured to be rapidly used in downstream processes such as immunofluorescence or flow cytometry assays. Furthermore, it is known in the art that the genotoxicity of colibactin was inhibited when bacteria and mammalian cells were separated by a porous membrane, indicating that colibactin genotoxicity is contact-dependent [3]. Without wishing to be bound by any particular theory, it is thought that Matrigel might act as a physical barrier between both cell types and partially prevent colibactin-dependent genotoxicity. Therefore, performing the first and the second phases in suspension is preferred as it allows the bacteria and organoid or organoid fragments to be in direct contact. Therefore, in some embodiments, the first phase is conducted in suspension culture. In some embodiments, the second phase is conducted in suspension culture.

In some embodiments, the methods for co-culturing genotoxic bacteria and organoids do not involve injecting the genotoxic bacteria into the lumen of the organoid. The first phase, the second phase and/or the third phase may not involve injecting the bacteria into the lumen of the organoid. As set out in detail below, the methods of the invention involve co-culturing genotoxic bacteria and organoids so that the genotoxic bacteria can either interact with the apical surface of the organoid fragments or the basal surface of the organoids. In some embodiments, the first phase, the second phase and/or the third phase do not involve culturing the genotoxic bacteria with the apical surface of the organoid.

The invention also provides methods of producing an organoid with a mutational signature, using the phases described herein. For example, the invention provides a method of producing an organoid with a mutational signature wherein the method comprises at least two phases selected from a first phase comprising culturing organoids or organoid fragments with genotoxic bacteria; a second phase comprising culturing the organoids or organoids fragments with genotoxic bacteria, wherein the bacterial growth is restricted with antibiotics, a third phase comprising killing the genotoxic bacteria and enabling the further growth of the organoids or organoid fragments, wherein an organoid or an organoid fragment that has a mutational signature is produced.

### The First Phase

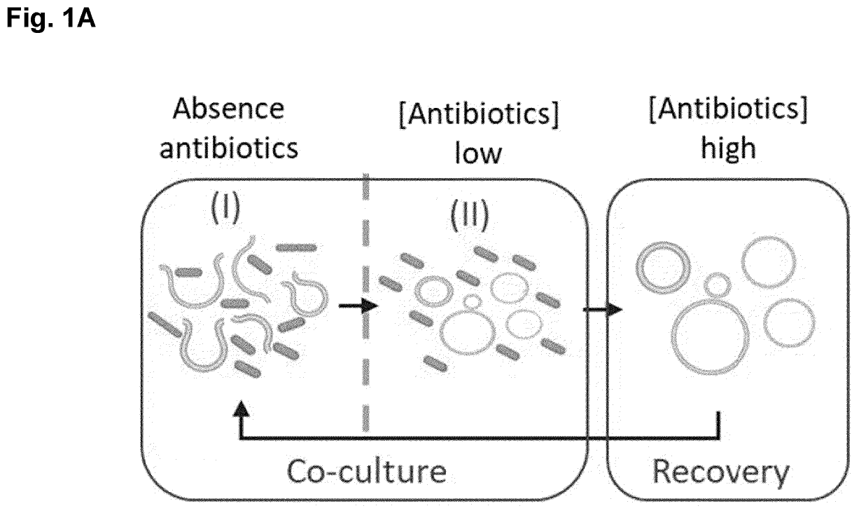

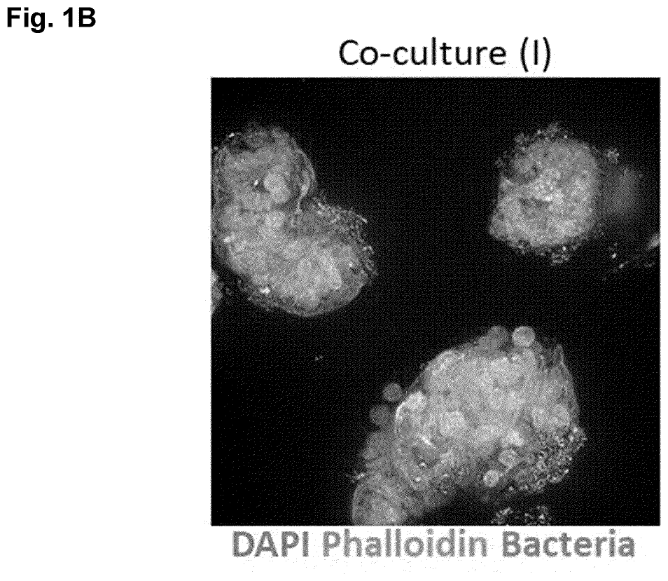

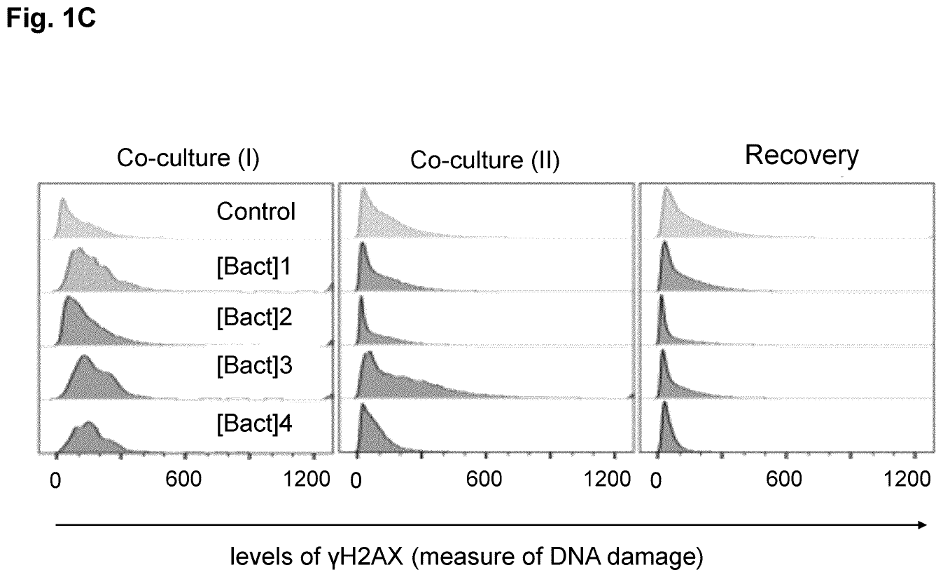

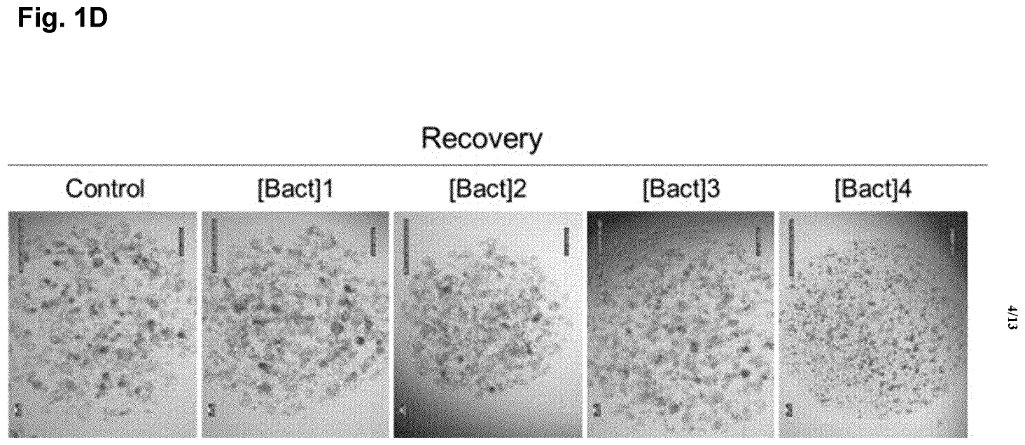

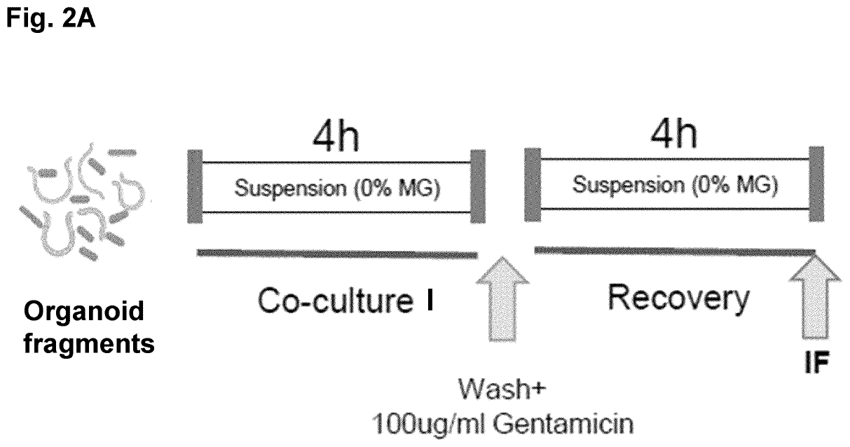

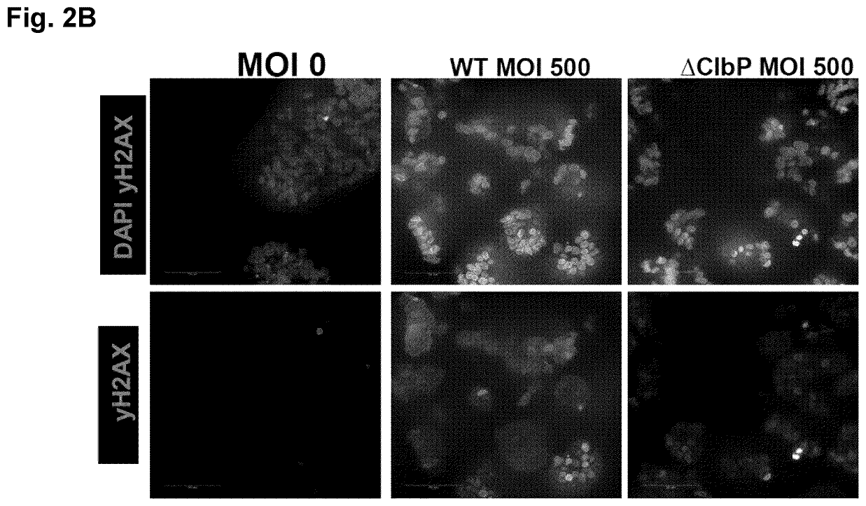

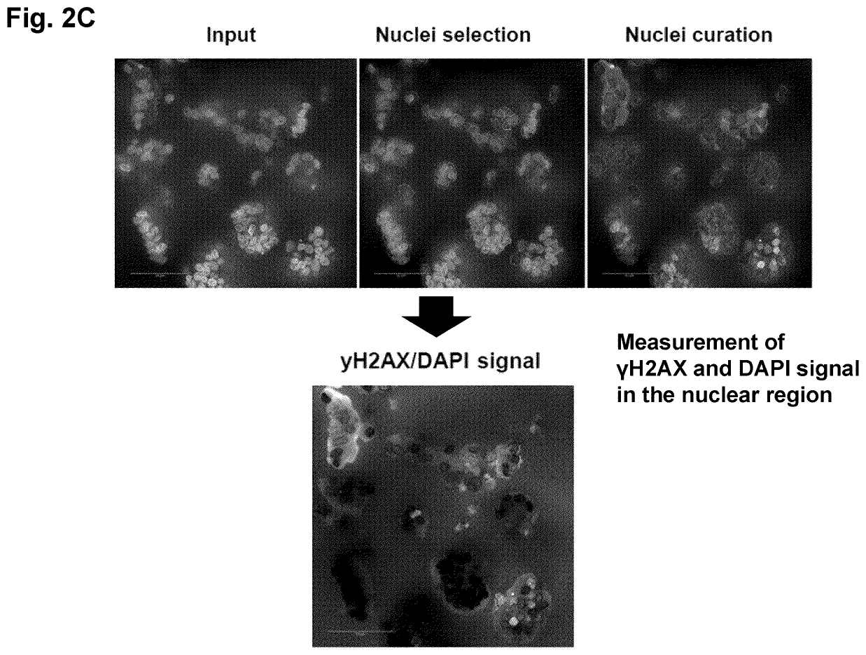

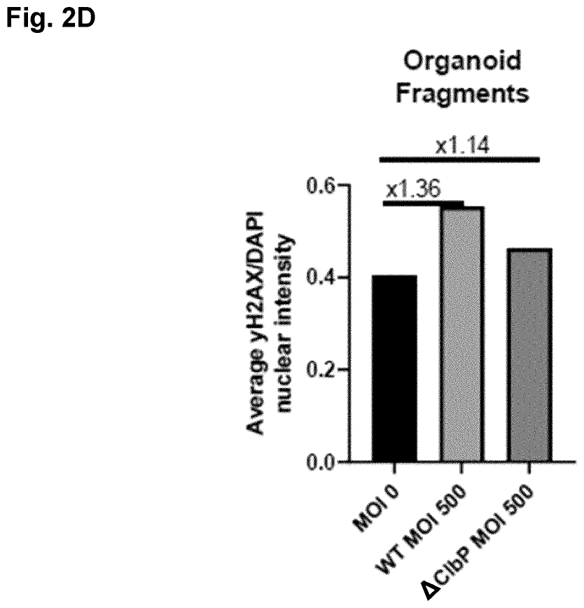

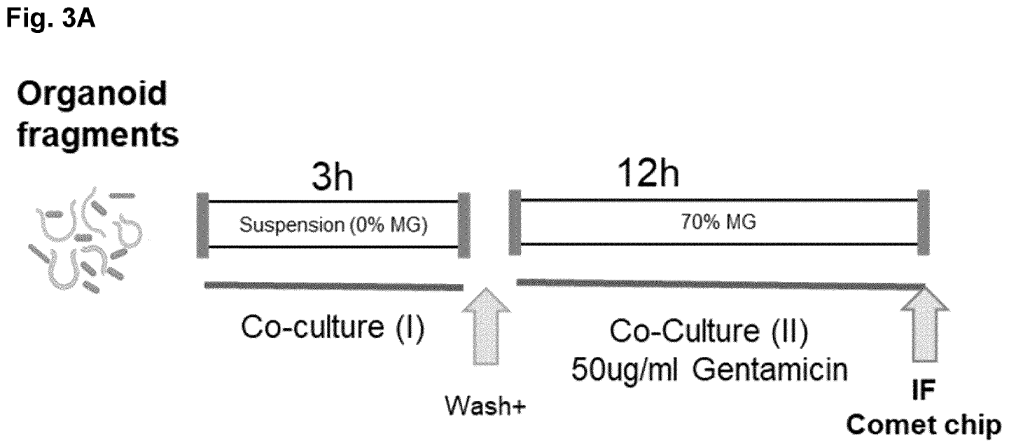

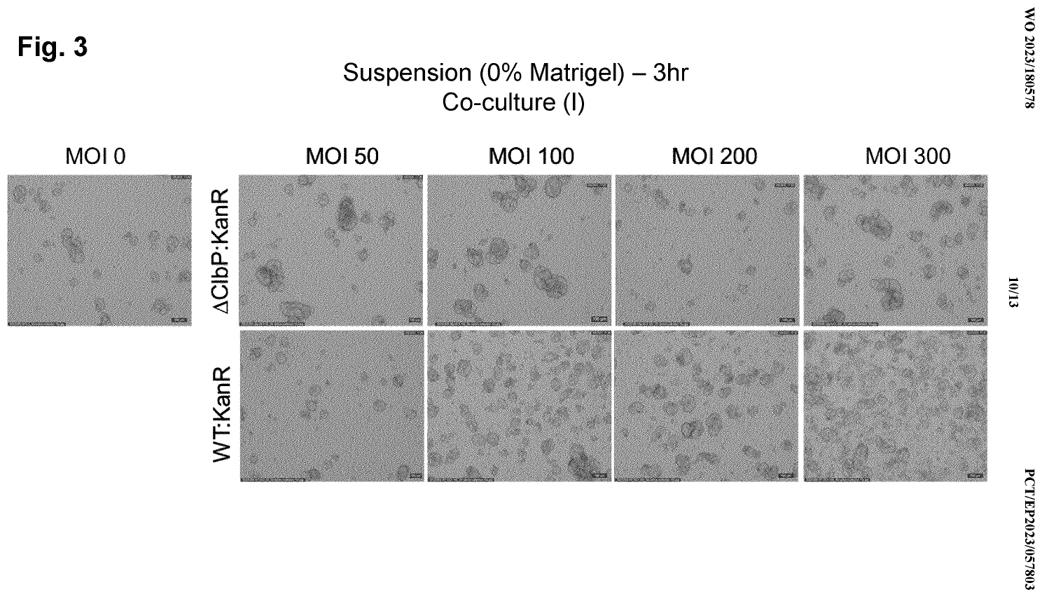

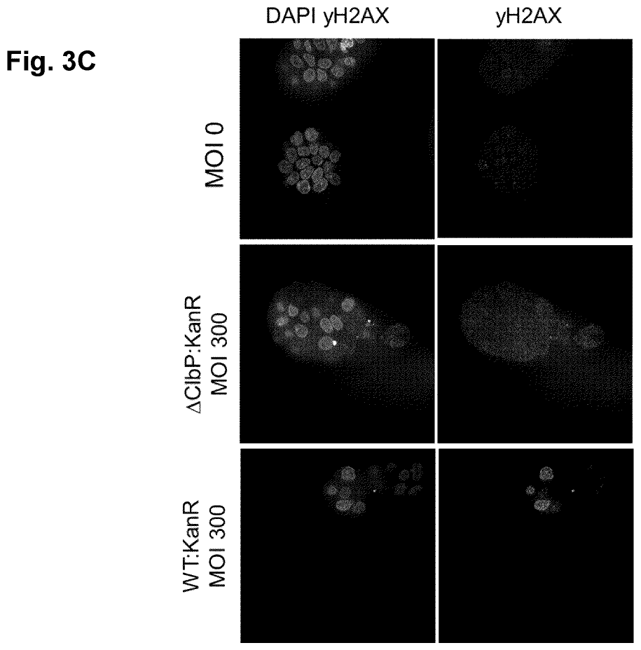

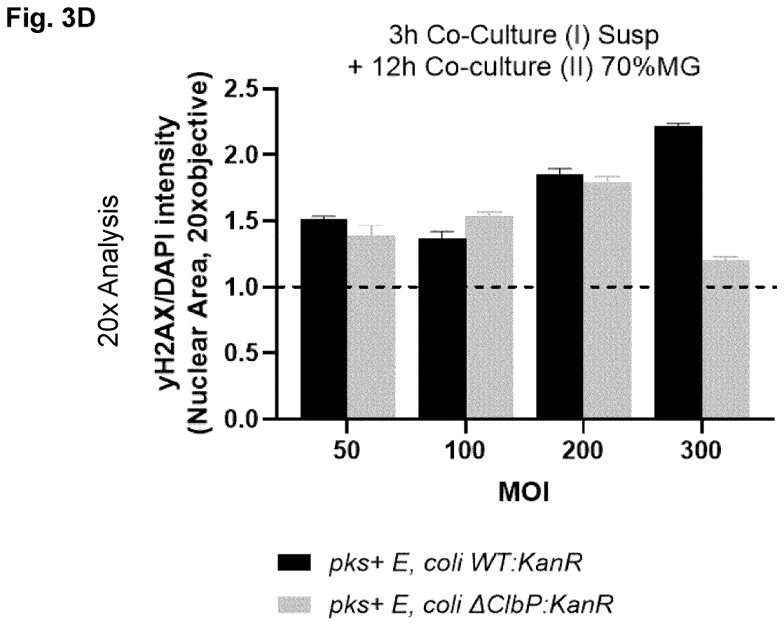

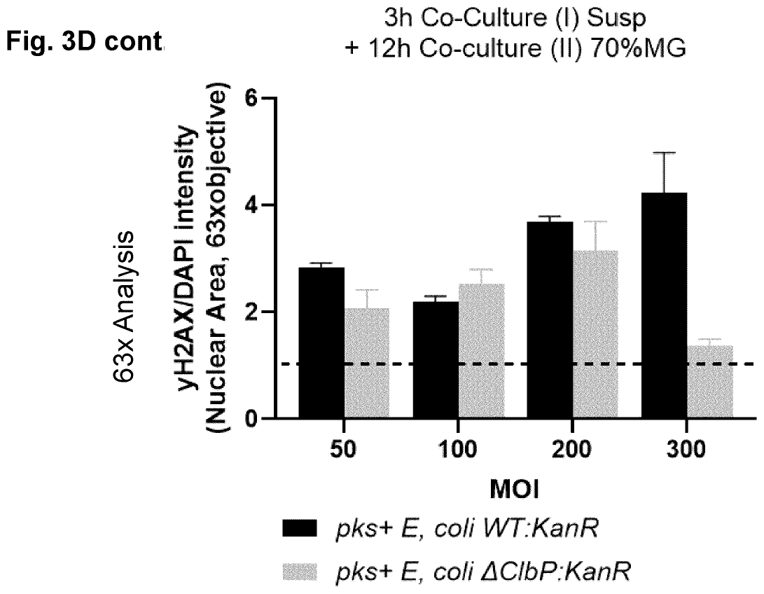

The first phase comprises culturing organoids or organoid fragments with genotoxic bacteria. The purpose of the first phase is to bring the bacteria and the organoid cells into close proximity in the culture and/or produce a large amount of DNA damage (“acute damage”). For example, in a long term exposure co-culture strategy scheme, that involves the first phase, the second phase and the third phase (an example of this scheme is provided in FIG. 1A), the aim of the first phase is produce a large amount of DNA damage in the organoids or organoid fragments. In an alternative embodiment, a rapid co-culture strategy scheme, that involves the first phase and the third phase (an example of this scheme is provided in FIGS. 2A and 3A), the aim of the first phase is bring the bacteria and the organoid cells into close proximity in the culture.

Acute damage can be defined as damage that results in significant loss of cell viability. For example, acute damage can result in at least 30%, at least 40%, at least 50%, at least 60%, at least 70%, at least 80%, at least 90% or 100% loss in cell viability. In some embodiments, after the first phase at least 50% of the organoids and organoid fragments are viable. For example, at least 50%, at least 60% or at least 70% of the organoids and organoid fragments are viable after the first phase.

Various assays are known in the art to measure cell viability including assays that determine viability based on cellular membrane integrity (e.g. using nucleic acid dyes Propidium iodide, TO-PRO-3 lodide or 7-AAD), a cellular function such as enzymatic activity (e.g. using Calcein or a CyQUANT Cytotoxicity Assay Kit) or metabolic activity (e.g. using alarmarBlue Cell Viability Reagent or a yQUANT MTT Cell Viability Assay). In some embodiments, at least 1%, at least 2%, at least 3%, at least 4%, at least 5%, at least 7.5%, at least 10%, at least 20%, at least 30% or at least 40% of the DNA in the organoid or organoid fragment is damaged e.g. as measured by the Comet assay [4] or a yH2AX assay [5] (e.g. as shown in FIG. 2C).

The first phase can comprise culturing organoids, organoid fragments or a combination of organoid fragments and organoids.

The apical surface of intestinal epithelial cells faces the lumen and regulates interactions with lumenal contents, including mediating nutrient absorption, detects microbial products, and secretes molecules that protect the epithelium from potentially harmful agents in the lumen. The basolateral surface anchors epithelial cells to the underlying basement membrane, delivers nutrients from the lumen to the bloodstream, and communicates with nearby cells [6]. As discussed in detail below, polarised organoids have an apical surface on the inside of the organoid and a basal surface on the outside of the organoid. It is generally believed that genotoxic bacteria need to interact with the apical surface of the organoid in order to damage the DNA. Fragmenting the organoids allows the apical surface to be exposed to genotoxic bacteria in the co-culture. Therefore in some embodiments, the first phase comprises culturing organoid fragments. Organoid fragments for use in the first phase can be produced by shearing one or more organoids, for example with a glass pipette. This can involve first enzymatically digesting the one or more organoids and then shearing them. In some embodiments, the method comprises obtaining organoid fragments by shearing one or more organoids and then culturing the organoid fragments with genotoxic bacteria. In some embodiments, the organoids to be sheared are grown in 60-80% Matrigel, optionally about 70% Matrigel, for 2-4 days.

The organoid fragments naturally reform organoids in the culture. This reformation can occur at any phase in the method, but primarily occurs during the second or third phase of the method.

The present inventors have, for the first time, hypothesised that apical exposure to genotoxic bacteria is not necessary for DNA damage, and that exposure (particularly longer-term exposure) of the basal surface of the organoid (optionally without fragmentation) can result in DNA damage. Therefore, in other embodiments, the first phase (and later phases) comprises culturing organoids (without fragmentation) with genotoxic bacteria which results in DNA damage.

It is well known in the art that the growth of bacteria in batch culture can have four phases, lag phase, exponential phase (as known as log phase), stationary phase and death phase. During lag phase, bacteria adapt themselves to growth conditions and there is little to no cell division. The exponential phase is a period characterized by cell doubling. The number of new bacteria appearing per unit time is proportional to the present population. If growth is not limited, doubling will continue at a constant rate so both the number of cells and the rate of population increase doubles with each consecutive time period. The stationary phase is often due to a growth-limiting factor such as the depletion of an essential nutrient, and/or the formation of an inhibitory product such as an organic acid. Stationary phase results from a situation in which growth rate and death rate are equal. At death phase (decline phase), bacteria die, which can be caused by lack of nutrients, environmental temperature above or below the tolerance band for the species. The skilled person is aware of different techniques to determine the bacterial growth rate, including measuring the optical density (OD) at 600 nm (OD600). Optical density measures the degree of light scattering caused by the bacteria within a culture; the more bacteria there are, the more the light is scattered. By monitoring the rate of increase in OD600, you can identify the lag, log, and stationary phases of a bacterial culture. Assays for measuring the bacterial growth rate using OD600 are well known in the art and are described in refs [27], and [31].

In some embodiments, the genotoxic bacteria is in an exponential growth phase when it is incorporated into the co-culture. In some embodiments, the genotoxic bacteria in an exponential growth phase can have an optional density measured at a wavelength of 600 nm (OD600) of >0.4 and <1. It is preferably to incorporate genotoxic bacteria into the co-culture that is in an exponential growth phase as this ensures the fitness of the bacteria introduced in the system which improves the consistency between the replicates of the claimed methods.

The multiplicity of infection (MOI) is the ratio of genotoxic bacteria to organoids or organoid fragments. The MOI of the bacteria can be determined using colony forming unit assay as described in refs [27], and [31]. In some embodiments, in the first phase the genotoxic bacteria has a multiplicity of infection (MOI) of 0.1, 0.5, 1, 2, 5, 10, 20, 30, 40, 50, 100, 200, 300, 400, 500, 600, 700 or 800 when it is incorporated into the co-culture. In preferred embodiments, the genotoxic bacteria has a multiplicity of infection of between 0.1 and 800, between 10 and 100, between 50 and 100, or about 100 when it is incorporated into the co-culture. In preferred embodiments, in the first phase the genotoxic bacteria has an MOI of 10, 50 or 100. In preferred embodiments, in the first phase the genotoxic bacteria has an MOI of 10, 50 or 100 and the genotoxic bacteria is in an exponential growth phase can have an optional density measured at a wavelength of 600 nm (OD600) of >0.4 and <1. In preferred embodiments, in the first phase the genotoxic bacteria has an MOI of 10, 50 or 100 and is cultured for 3 to 4 hrs. In preferred embodiments, in the first phase the genotoxic bacteria has an MOI of 500 and is cultured for 4 hours.

The length of the first phase can vary, e.g. depending on the growth rate of the bacteria, the length of the second phase and the amount of acute damage desired. For example, the first phase can occur for between 15 mins to 5 hours. In some embodiments, the first phase can occur for between 30 mins to 18 hours. In some embodiments, the first phase occurs for 30 mins to 5 hrs, 1 hr to 4 hrs, 1 hr to 3 hrs, 2 hrs to 4 hrs, 2 hrs to 4 hrs or 3 hrs to 4 hrs. In some embodiments, the first phase occurs for at least 30 mins, at least 1 hr, at least 1.5 hrs, at least 2 hrs, at least 2.5 hrs, at least 3 hrs, at least 3.5 hrs, at least 4 hrs, at least 4.5 hrs, at least 5 hr. In preferred embodiments, the first phase occurs for at least 3 hrs. In preferred embodiments, the first phase occurs for 3 to 4 hrs.

The quantity of bacteria added to the culture in the first phase (i.e. the multiplicity of infection) and the length of the first phase can be related. In general, if a higher MOI is used when the bacteria is incorporated into the co-culture then the length of the first phase can be shorter. If a low MOI is used when the bacteria is incorporated into the co-culture then the first phase can occur for a longer time. For example, if a MOI of less than 50 is used then the first phase can occur overnight (e.g. at least 12 hours). In some embodiments, the first phase occurs for between 15 mins to 5 hours and the genotoxic bacteria has a multiplicity of infection of 100 to 500 when it is incorporated into the co-culture. In some embodiments, the first phase occurs for between 3 mins to 6 hours and the genotoxic bacteria has a multiplicity of infection of 50 to 200 when it is incorporated into the co-culture. In some embodiments, the first phase occurs for between 6 to 8 hours and the genotoxic bacteria has a multiplicity of infection of 0.1 to 50 when it is incorporated into the co-culture.

The first phase can occur in the absence of an antibiotic and/or the growth of the bacteria is unrestricted.

### The Second Phase

The second phase can occur after the first phase, or in alternative embodiments, the second phase is the first step in the method, for example in a method that comprises the second and the third phase.

In the second phase, the growth of genotoxic bacteria is restricted with one or more antibiotics. In this phase the growth of the organoids or organoid fragments is not restricted. Many antibiotics are well known in the art. Exemplary antibiotics for use in the methods of the invention include Ampicillin, Carbenicillin, Fosmidomycin, Gentamicin, Amphotericin, Kanamycin, Neomycin, Primocin, Penicillin & Streptomycin (PenStrep), cationic antimicrobial peptides (CAMPs) such as polymixin B, tetracyclin and Chloramphenicol, and Streptomycin. In preferred embodiments, the antibiotic is Gentamicin. The purpose of the antibiotic in the second phase is to restrict the growth of the bacteria rather than to kill it. The amount of antibiotic used in the second phase will depend on the antibiotic employed in the method, the bacteria strain and the bacterial MOI used. For example, the second phase can comprise 0.1-100 μg/ml of gentamicin. In some embodiments, the second phase comprises about 50 μg/ml of gentamicin. Suitable antibiotic concentrations to restrict the growth of genotoxic bacteria can easily be identified by the skilled person by testing the growth rate of the genotoxic bacteria with varying concentrations of antibiotic.

The restricted genotoxic bacteria will no longer have a classical growth phases. In the presence of low concentrations of antibiotics the bacteria are expected to follow a ‘bistable growth’ pattern as described in [7]. Bacteria bistable growth is characterized by the coexistence of growing and nongrowing populations.

The restricted genotoxic bacteria will have a decreased doubling time or follow a bistable growth with a plateau-like fitness landscape. In some embodiments, the doubling time of the genotoxic bacteria in the second phase can be reduced by at least 10%, 20%, 30%, 40% or 50%. If the method comprises the first and the second phases, then the growth rate of the genotoxic bacteria in the second phase follow a bistable growth and/or the growth rate is lower than the growth rate of the genotoxic bacteria in the first phase. For example, the growth rate of the genotoxic bacteria in the second phase can be reduced by at least 10%, 20%, 30%, 40%, 50%, 60%, 70% or 80% compared to the growth rate of the genotoxic bacteria in the first phase. If the second phase is the first phase in the method, then the growth of the rate of the genotoxic bacteria in the second phase is lower compared to a culture medium which does not contain an antibiotic and/or the growth of the bacteria is unrestricted. In some embodiments, the growth rate of the genotoxic bacteria in the second phase is lower compared to the growth of the genotoxic bacteria in a medium that does not contain an antibiotic and/or the growth of the bacteria is unrestricted. It is well known in the art how to compare the growth rates of different bacteria.

The length of the second phase can vary, e.g. depending on the length of the first phase, the amount of DNA damage induced in the first phase, the bacteria load at the end of the first phase, the required organoid cell viability and the amount of sustained damage desired. For example, the second phase can occur for between 0.5 days to 3 days. In some embodiments, the second phase occurs for at least 0.5 days, at least 1 day, at least 1.5 days, at least 2 days, at least 2.5 days or at least 3 days. In some embodiments, the second phase can occur for between 1 day to 3 days, 1 day to 2 days or 2 days to 3 days. In preferred embodiments, the second phase is 2 days long. If the first phase has been short, for example 15 mins to 1 hr, the second phase is preferably longer, for example 2 to 3 days.

The second phase can occur after the first phase, or in alternative embodiments, the second phase is the first step in the method. If the second phase occurs after the first phase then between the first and the second phase the organoids or organoid fragments can be washed. This washing step can involve washing the organoids or organoid fragments with a saline solution (i.e. PBS) or a mammalian cell medium (i.e. AdDMEM) for 1-5 min at a speed <500 G. Mammalian cells and organoids precipitate at slower centrifugal speeds, such as <500G, whereas bacteria cells require higher centrifugal speeds to be precipitated, such as approximately 3000G. This difference in centrifugal precipitation speeds allows the organoids to be separated from the majority of the bacteria. This washing step enables the reduction and control of the bacteria load when transferring from the first to the second phase. In other embodiments, the organoids in the first phase are treated such that all the bacteria in the culture is removed, for example by treating the first phase with a high concentration of an antibiotic (such as at least 150 μg/ml, at least 180 μg/ml, at least 190 μg/ml, at least 200 μg/ml of gentamicin, optionally about 200 μg/ml of gentamicin), and then replenishing the culture with further genotoxic bacteria.

If the second phase is the first step in the method then genotoxic bacteria needs to be incorporated into the co-culture. In some embodiments, the genotoxic bacteria is in an exponential growth phase when it is incorporated into the co-culture. In some embodiments, the genotoxic bacteria in an exponential growth phase can have an optional density measured at a wavelength of 600 nm (OD600) of >0.4 and <1. It is preferable to incorporate genotoxic bacteria into the co-culture that is in an exponential growth phase as this ensures that the bacteria growth throughout the phase remains essentially constant which improves the consistency between the replicates of the claimed methods. The multiplicity of infection (MOI) is the ratio of genotoxic bacteria to organoids or organoid fragments. The MOI of the bacteria can be determined using colony forming unit assay as described in refs [27], and [31]. In some embodiments, in the first phase the genotoxic bacteria has a multiplicity of infection (MOI) of 0.1, 0.5, 1, 2, 5, 10, 20, 30, 40, 50, 100, 200, 300, 400, 500, 600, 700 or 800 when it is incorporated into the co-culture. In preferred embodiments, the genotoxic bacteria has a multiplicity of infection of between 0.1 and 800, between 10 and 100, between 50 and 100, or about 100 when it is incorporated into the co-culture.

During the second phase the organoid fragments can form one or more organoids, for example in some embodiments the during the second phase at least 60%, at least 70%, at least 80% or at least 90% of the organoid fragments form one or more organoids. In some embodiments, the organoids that are formed can be polarised. As explained in detail below, polarised organoids have an apical surface on the inside of the organoid and a basal surface on the outside of the organoid. Without wishing to be bound by any particular theory, it is believed that the apical surface does not necessarily need to be exposed for the DNA in the organoid to be damaged. Therefore, during the second phase, when the organoid fragments can form one or more organoids, the genotoxic bacteria will exposed to the basal surface of the organoid. The inventors hypothesize that the DNA damage caused by genotoxic compounds on the basal surface can produce mutational signatures that are similar to human cancer signatures.

If the method comprises a first phase and a second phase only then the method can produce an organoid or an organoid fragment that has a mutational signature.

After the second phase at least 20% of the organoids and organoid fragments are expected to be viable. For example, at least 20%, at least 25% or at least 30% of the organoids and organoid fragments are viable after the second phase.

### The Third Phase

The third phase can occur after the first or the second phase, for example the third phase can occur after the first phase only, after the first phase then the second phase or after the second phase only.

In the third phase the genotoxic bacteria in the culture is removed, for example by killing the bacteria with a high concentration of an antibiotic. In this phase the growth of the organoids or organoid fragments is not restricted. Exemplary antibiotics for use in the third phase include Ampicillin, Carbenicillin, Fosmidomycin, Gentamicin, Amphotericin, Kanamycin, Neomycin, Primocin, Penicillin & Streptomycin (PenStrep), cationic antimicrobial peptides (CAMPs) such as polymixin B, tetracyclin and Chloramphenicol, and Streptomycin. In preferred embodiments, the antibiotic is Gentamicin. The amount of antibiotic used in the third phase will depend on the antibiotic employed in the method, the bacteria strain and the bacterial MOI used. In some embodiments, the genotoxic bacteria are killed with 100-300 μg/ml of gentamicin, optionally the genotoxic bacteria are killed with 200 μg/ml of gentamicin. In some embodiments, the genotoxic bacteria are killed with 20-100 μg/ml of Primocin, optionally the genotoxic bacteria are killed with 50 μg/ml of Primocin.

After the DNA has been damaged by the genotoxic bacteria the organoid or organoid fragment can repair the damage, which can result in mutational errors in the DNA. Following DNA damage the cells may enter into G2/M cell cycle arrest. Only cells that re-enter into proliferation are predicted to have resolved the DNA damage by incorporating mutations. This repair can occur at any point in the method by predominantly occurs in the third phase. During the third phase the organoids or organoid fragments can recover from the DNA damage and/or expand. The purpose of the third phase is to obtain sufficient material for downstream processes and/or to ensure that the cells are healthy enough to survive any further rounds of DNA damage.

At the end of the recovery period (i.e. at the end of the third phase) less than 60% of the organoid or organoid fragments are expected to be arrested in G2/M. Alternatively, more than 5% of the organoid or organoid fragments are positive for ki67 staining (a marker for cell proliferation) after the third phase.

The length of the third phase can vary, e.g. according to the recovery time required, the amount of genotoxic bacterial exposure or the type of organoid cultured. For example, the third phase can occur for between 2 days to 20 days hours. In some embodiments, the third phase occurs for at least 0.5 days, at least 2 days, at least 3 days, at least 4 days, at least 5 days, at least 6 days, at least 7 days, at least 8 days, at least 9 days, at least 10 days, at least 12 days, at least 14 days, at least 16 days, at least 18 days or at least 20 days. In some embodiments, the third phase can occur for between 2 days to 15 days, 5 day to 15 days, 10 days to 15 days. For some organoid cultures, such as lung organoids which take longer to passage, the third phase can be longer, for example the third phase can be 15 to 25 days or 15 days to 20 days. In some embodiments, the third phase occurred for 2-5 days.

In some embodiments, the third phase is conducted in suspension rather than on a substrate, such as Matrigel. This enables the organoids that are being cultured in the medium to be rapidly used in downstream processes such as immunofluorescence or flow cytometry assays.

During the third phase the organoid fragments can form one or more organoids, for example in some embodiments the during the third phase at least 60%, at least 70%, at least 80% or at least 90% of the organoid fragments form one or more organoids.

After the third phase, the method can produce an organoid or an organoid fragment that has a mutational signature.

Exemplary methods for co-culturing genotoxic bacteria and organoids include:

A method for co-culturing genotoxic bacteria and organoids, wherein the method comprises a first phase comprising culturing organoids with genotoxic bacteria and a second phase comprising culturing the organoids with genotoxic bacteria, wherein the bacterial growth is restricted with antibiotics.

A method for co-culturing genotoxic bacteria and organoid fragments, wherein the method comprises a first phase comprising culturing organoid fragments with genotoxic bacteria and a second phase comprising culturing the organoids fragments with genotoxic bacteria, wherein the bacterial growth is restricted with antibiotics. In this embodiment, the organoid fragments can naturally reform organoids in the culture in any phase, but this primarily occurs during the second phase of the method.

A method for co-culturing genotoxic bacteria and organoids, wherein the method comprises a second phase comprising culturing the organoids with genotoxic bacteria, wherein the bacterial growth is restricted with antibiotics and a third phase comprising killing the genotoxic bacteria and enabling the further growth of the organoids.

A method for co-culturing genotoxic bacteria and organoid fragments, wherein the method comprises a second phase comprising culturing the organoids fragments with genotoxic bacteria, wherein the bacterial growth is restricted with antibiotics, a third phase comprising killing the genotoxic bacteria and enabling the further growth of the organoid fragments. In this embodiment, the organoid fragments can naturally reform organoids in both the second and third phases.

A method for co-culturing genotoxic bacteria and organoids, wherein the method comprises a first phase comprising culturing organoids with genotoxic bacteria and a third phase comprising killing the genotoxic bacteria and enabling the further growth of the organoids.

A method for co-culturing genotoxic bacteria and organoid fragments, wherein the method comprises a first phase comprising culturing organoid fragments with genotoxic bacteria and a third phase comprising killing the genotoxic bacteria and enabling the further growth of the organoid fragments. In this embodiment, the organoid fragments can naturally reform organoids in the culture in any phase, but this primarily occurs during the third phase of the method.

A method for co-culturing genotoxic bacteria and organoids, wherein the method comprises a first phase comprising culturing organoids with genotoxic bacteria; a second phase comprising culturing the organoids with genotoxic bacteria, wherein the bacterial growth is restricted with antibiotics, and a third phase comprising killing the genotoxic bacteria and enabling the further growth of the organoids.

A method for co-culturing genotoxic bacteria and organoid fragments, wherein the method comprises a first phase comprising culturing organoid fragments with genotoxic bacteria; a second phase comprising culturing the organoids fragments with genotoxic bacteria, wherein the bacterial growth is restricted with antibiotics, and a third phase comprising killing the genotoxic bacteria and enabling the further growth of the organoid fragments. In this embodiment, the organoid fragments can naturally reform organoids in any phase, but this primarily occurs during the second and third phases.

While the above embodiments set out methods of co-culturing genotoxic bacteria with organoids or organoid fragments, as the organoid fragments can naturally reform organoids in the culture at any time, all of the phases can contain a mixture of organoids or organoid fragments.

### Bacteria

The human microbiome is the aggregate of all microbiota that reside on or within human tissues and biofluids, including the skin, mammary glands, seminal fluid, uterus, ovarian follicles, lung, saliva, oral mucosa, lacrimal tissue, conjunctiva, biliary tract, retinal tissue, and gastrointestinal tract. The gastrointestinal tract includes the mouth, the pharynx (throat), the esophagus, the stomach, the small intestine, the large intestine, the colon, the rectum and the anus. Some of the bacteria in the microbiome are genotoxic bacteria. Genotoxic bacteria are any bacteria that secretes a bacterial genotoxin. Bacterial genotoxins are a class of molecules that have the ability to enter the nucleus of a host cell and cause DNA damage by introducing single- and double-strand DNA breaks, leading to various effects, including activation of DNA damage response, senescence, apoptosis, and genetic aberrations [8]. The bacteria used in the methods of the invention are genotoxic bacteria and are used to produce organoids or organoid fragments that have a mutational signature.

In some embodiments, the genotoxic bacteria is a colibactin-producing bacteria. Colibactin is a natural genotoxic compound which is synthetized by polyketide synthases, non-ribosomal peptide synthases, and hybrid enzymes encoded by a 54-kb genomic island designated pks. The pks island is found in members of the family Enterobacteriaceae, including Escherichia coli, Klebsiella pneumoniae, Enterobacter aerogenes and Citrobacter koseri. Colibactin induces DNA double-strand breaking, chromosome aberrations, and cell cycle arrest in the G2/M phase [1]. In some embodiments, the colibactin-producing bacteria is from the family Enterobacteriaceae. In some embodiments, the colibactin-producing bacteria is from the species Escherichia coli, Klebsiella pneumoniae, Enterobacter aerogenes and Citrobacter koseri. In preferred embodiments, the colibactin producing bacteria is from the species E. coli. In further preferred embodiments, the colibactin producing bacteria pks+E. coli.

The genotoxic bacteria can also be a bacterium that has been genetically engineered to express a genotoxic compound, such as a pks genomic island.

Alternative genotoxic bacteria include Non-Typhoidal Salmonellae, Campylobacter jejuni, Fusobacterium nucleatum, Helicobacter pylori, Shigella, E. faecalishttps://pubmed.ncbi.nlm.nih.gov/23661025/or Akkermansia muciniphila.https://pubmed.ncbi.nlm.nih.gov/23661025/ORGANOIDS

The invention relates to methods for co-culturing genotoxic bacteria and organoids. Organoids can be obtained by culturing epithelial stem cells. The epithelial stem cells are obtained from adult tissue, i.e. the epithelial stem cells are adult epithelial stem cells. In this context “adult” means mature tissue, i.e. includes newly-born baby or child but excludes embryonic or foetal. Alternatively, the epithelial stem cells are not derived from embryonic stem cells or embryonic stem cell lines, e.g. which have been differentiated in vitro. The adult tissue may be healthy adult tissue. In other embodiments, the adult tissue may be from a patient with a disease genotype or phenotype, such as cancer or an inflammatory disease.

The epithelial stem cell may be derived from colorectal, small intestine, stomach, pancreas, liver, lung, breast, prostate, kidney, mouth, nasopharynx, throat, hypopharynx, larynx, trachea, skin, fallopian tube, ovary, salivary gland, esophagus, hair follicle and/or cochlear tissue. In some embodiments the epithelial stem cells are colorectal cells. Methods for culturing epithelial stem cells to obtain organoids from a variety of epithelial tissues have previously been described (e.g. in WO2009/022907, WO2010/090513, WO2012/014076, WO2012/168930, WO2015/173425, WO2016/083613, and WO2016/083612, WO2017/149025, WO2020/234250 and [9]). Cells taken directly from tissue, i.e. freshly isolated cells, are also referred to as primary cells. In some embodiments the epithelial stem cells are primary epithelial stem cells.

As mentioned above, bacteria can reside on or within various human tissues and biofluids. These tissues are particularly relevant for the co-culture methods of the invention. Therefore, in some embodiments, the organoid fragment or organoid for use in the method can therefore be derived from the gastrointestinal tract, pancreatic tissue, lung tissue, the trachea, the skin and/or vaginal tissue. In preferred embodiments, the organoid fragment or organoid is derived from the gastrointestinal tract. The gastrointestinal tract includes the mouth, the pharynx (throat), the esophagus, the stomach, the small intestine, the large intestine, the colon, the rectum and the anus. Therefore, in some embodiments, the organoid fragment or organoid is derived from the mouth, the pharynx (throat), the esophagus, the stomach, the small intestine, the large intestine, the colon, the rectum and the anus. In preferred embodiments, the organoid fragment or organoid is derived from stomach, the small intestine, the large intestine, the colon or the rectum. In preferred embodiments, the organoid fragment or organoid is derived from colon tissue.

The organoid with a mutational signature produced by the claimed methods can be a gastrointestinal organoid, a pancreatic organoid, a lung organoid, a trachea organoid, a skin organoid or a vaginal organoid.

An organoid used in the present invention is preferably obtained using an epithelial cell from an adult tissue, optionally an epithelial stem cell from an adult tissue expressing Lgr5. In some embodiments, an organoid originates from a single cell, optionally expressing Lgr5. Advantageously, this allows a homogenous population of cells to form. A single cell suspension comprising the epithelial stem cells can be mechanically generated. In some embodiments, the single cell suspension comprising the epithelial stem cells is generated using mechanical processes and/or enzymatic digestion. Mechanical processes include, but are not limited to dissection, micro-dissection and filtering.

An organoid obtained using a culture medium suitable for expansion may be referred to as an “expansion organoid”. An expansion organoid comprises at least one epithelial stem cell, which can divide and produce further epithelial stem cells or can generate differentiated progeny. It is to be understood that in a preferred expansion organoid, the majority of cells are expanding cells (i.e. dividing cells) that retain an undifferentiated phenotype. Although some spontaneous differentiation may occur, the cell population is generally an expanding population. The length of time that the organoids can continue to be expand whilst maintaining a core presence of epithelial stem cells and whilst maintaining genotypic and phenotypic integrity of the cells, is an important feature of the organoids. Organoids typically express Lgr5. The organoids also have a distinctive structure that arises rapidly as the cells expand and self-organise in vitro. These features are described in detail below.

Image analysis may be used to assess characteristics of cells in culture such as cell morphology; cell structures; evidence for apoptosis or cell lysis; and organoid composition and structure. Many types of imaging analysis are well known in the art, such as electron microscopy, including scanning electron microscopy and transmission electron microscopy, confocal microscopy, stereomicroscopy, fluorescence microscopy. Histological analysis can reveal basic architecture and cell types.

In some embodiments, the organoid has a three dimensional structure, i.e. the organoid is a three-dimensional organoid. In some embodiments, the organoid comprises epithelial cells. In some embodiments, the organoid comprises only epithelial cells, i.e. non-epithelial cells are absent from the organoid. Even if other cell types are transiently present in the culture medium, e.g. in the tissue fragment that is the starting material, these cells are unlikely to survive and instead will be replaced by the longer term expansion of the stem cells which generate a pure population of epithelial cells.

In some embodiments, the epithelial cells in the organoid surround a lumen. In some embodiments, the organoid does not comprise a lumen (in particular the tumour organoids generally do not have a lumen). In some embodiments, the epithelial cells are polarized, (meaning that proteins are differentially expressed on the apical or basolateral side of the epithelial cell). In some embodiments the lumen is a sealed lumen (meaning that a continuous cellular barrier separates the contents of the lumen from the medium surrounding the organoid). In some embodiments the organoids comprise stem cells which are able to actively divide and which are preferably able to differentiate to all major differentiated cell lineages present in the corresponding in vivo tissue, e.g. when the organoid or cell is transferred to a differentiation medium. In some embodiments, the organoid comprises basal cells on the outside and more differentiated cells in the centre.

In some embodiments, the organoids comprise stratified epithelium. By “stratified” it is meant that there are multiple (more than one) layers of cells. Such cells often tend to have their nuclei more central to the cells, i.e. not polarized. The cells in the multilayer section may organise themselves to include a gap, or lumen between the cells.

In some embodiments the organoids comprise single monolayers that are folded (or invaginated) to form two or more layers. It can sometimes be difficult to distinguish between folded (or invaginated) monolayers and regions of stratified cells. In some embodiments an organoid comprises both regions of stratified cells and regions of folded monolayers. In some embodiments the organoids have a section which is formed of multiple layers and a section comprising a single monolayer of cells. In some embodiments the organoids comprise or consist of a single monolayer of cells. In some embodiments, the organoid does not comprise a monolayer. The organoids may possess a layer of cells with at least one bud and a central lumen.

In some embodiments the organoids for use in the claimed methods comprise or consist of epithelial cells. In some embodiments, the organoids comprise or consist of a single layer of epithelial cells. In some embodiments non-epithelial cells are absent from the organoids.

In some embodiments, the organoid for use in the claimed methods has been cultured or is capable of culture in expansion media for at least 2 months, for example at least 10 weeks, at least 12 weeks, at least 14 weeks, at least 16 weeks, at least 4 months, at least 5 months, at least 6 months, at least 9 months, at least one year. In some embodiments, the organoid with a mutational signature produced by the claimed methods has been cultured or is capable of culture in expansion media for at least 2 months, for example at least 10 weeks, at least 12 weeks, at least 14 weeks, at least 16 weeks, at least 4 months, at least 5 months, at least 6 months, at least 9 months, at least one year.

In some embodiments, the organoid for use in the claimed methods or an organoid with a mutational signature produced by the claimed methods has been cultured or is capable of culture for at least 5 passages, at least 10 passages, at least 15 passages, or at least 20 passages. In some embodiments, the organoid or population of epithelial stem cells are cultured for at least 10 passages. In some embodiments, the organoid with a mutational signature produced by the claimed methods has been cultured or is capable of culture for at least 5 passages, at least 10 passages, at least 15 passages, or at least 20 passages. In some embodiments, the organoid or population of epithelial stem cells are cultured for at least 10 passages.

In some embodiments, the cell number of the organoid increases exponentially over at 5 passages, 10 passages, 15 passages, or 20 passages. In a preferred embodiment, the cell number of the organoid or population of epithelial stem cells increases exponentially over 5 passages.

In some embodiments, an organoid is at least 50 μm, at least 60 μm, at least 70 μm, at least 80 μm, at least 90 μm, at least 100 μm, at least 125 μm, at least 150 μm, at least 175 μm, at least 200 μm, at least 250 μm or more in diameter at the widest point.

Within the context of the invention, a tissue fragment is a part of an adult tissue, preferably a human adult tissue. An organoid, by contrast, develops structural features through in vitro expansion, and is therefore distinguished from a tissue fragment.

In a preferred embodiment, an organoid for use in the claimed methods or an organoid with a mutational signature produced by the claimed methods could be cultured during at least 2, 3, 4, 5, 6, 7, 8, 9, 10 weeks or 1, 2, 3, 4, 5, 6, 7, 8, 9, 10 months or longer. In some embodiments, the organoid is expanded or maintained in culture for at least 3 months, preferably at least 4 months, at least 5 months, at least 6 months, at least 7 months, at least 9 months, or at least 12 months or more.

### Mutational Signature

The DNA damage caused by the genotoxic bacteria results in a mutational signature in the organoid. The methods of the invention can therefore produce an organoid that has a mutational signature such as a cancer mutational signature. After being exposed to genotoxic bacteria in the methods of the invention, the organoids can be subcloned and analysed (e.g. by whole-genome sequencing or whole exome sequencing (WES)) to identify the resulting mutational signature. Cancer mutational signatures can be identified by comparing the signatures produced by the claimed methods to whole genome sequencing data from cancers.

As discussed above the human microbiome can exist in various organs in humans, including the skin, mammary glands, seminal fluid, uterus, ovarian follicles, lung, saliva, oral mucosa, conjunctiva, biliary tract, and gastrointestinal tract. A number of studies have indicated that dysfunction in the gut epithelial barrier can lead to the translocation of gut microbes to other organs in the human body. These translocated bacteria have been implicated in a number of cancer pathogeneses. For example, bacteria from the genera Bacteroides, Romboutsia and Lachnospiraceae have been identified in liver tumours [10]. Other studies show that Enterobacteriaceae and Pseudomonas bacteria are present in the pancreatic cancer tumor microenvironment [11]. The bacteria in the microbiome and translocated bacteria can therefore produce cancer mutational signatures in the relevant tissues.

In some embodiments, the methods of the invention produce an organoid that has a gastrointestinal tract mutational signature, such as a colorectal cancer mutational signature. In other embodiments, the methods of the invention produce an organoid that has a gastric cancer mutational signature, a pancreatic cancer mutational signature, a liver cancer mutational signature, a lung cancer mutational signature, an oral cavity cancer mutational signature, a nasopharynx cancer mutational signature, a tracheal cancer mutational signature, a skin cancer mutational signature, a vaginal cancer mutational signature, or an esophagus tissue cancer mutational signature.

For example, Pleguezuelos-Manzano, et al. demonstrated that repeated exposure of human intestinal organoids to genotoxic E. coli by repeated luminal injection over five months resulted in two mutational signatures. The first was a pks-specific single-base substitution signature where the organoids had an increased number single-base substitutions with a with a bias towards T>N substitutions (SBS-pks). These T>N substitutions occurred preferentially in ATA, ATT and TTT (with the middle base mutated). The second was a small indel signature (ID-pks), which was characterized by single T deletions at T homopolymers. Characterisations of the mutations in these two signatures identified the presence of an adenine residue 3 bp upstream of the mutated SBS-pks T>N site and in the ID-pks signature the poly-T stretches showed enrichment of adenines immediately upstream of the affected poly-T stretch. The lengths of the adenine stretch and the T-homopolymer were inversely correlated, consistently resulting in a combined length of five or more A/T nucleotides. Pleguezuelos-Manzano, et al. found that the SBS-pks and the ID-pks signatures were strongly enriched in CRC-derived metastases [2].

In some embodiments, the methods of the invention produce an organoid with an SBS-pks and an ID-pks mutational signature. In some embodiments, the methods of the invention produce an organoid with a single-base substitution signature. In some embodiments, the single-base substitution signature is characterised by an increased number single-base substitutions with a with a bias towards T>N substitutions. In some embodiments, the methods of the invention produce an organoid with a small indel signature. In some embodiments, the small indel signature results in an organoid that has an increased number single-base substitutions with a with a bias towards T>N substitutions.

### Culture Medium

Culture media suitable for culturing organoids and organoid fragments are well known in the art, e.g. as described in WO2009/022907, WO2010/090513, WO2012/014076, WO2012/168930, WO2015/173425, WO2016/083613, WO2016/083612, WO2017/149025 and WO2020/234250. The culture media mentioned in these documents are incorporated herein by reference and any of these may be used in the context of the invention.

In some embodiments, the culture medium suitable for culturing organoids and organoid fragments can comprise one or more of a Wnt agonist, a BMP inhibitor, a mitogenic growth factor and a TGF-beta inhibitor. For example, the culture medium suitable for organoids and organoid fragments comprises a Wnt agonist. The culture medium suitable for organoids and organoid fragments can further comprises a mitogenic growth factor and/or a BMP inhibitor. The same culture medium can be used in the first, the second and the third phase.

In preferred embodiments the culture medium suitable for organoids and organoid fragments comprises a Wnt agonist, a BMP inhibitor, a mitogenic growth factor and a TGF-beta inhibitor. The culture medium suitable for organoids and organoid fragments can further comprise one or more of a p38 inhibitor, a CAMP agonist, a prostaglandin pathway activator, nicotinamide, gastrin, B27 and N-acetylcysteine.

In some embodiments the culture medium comprises a basal medium for human or animal cells (such as DMEM/F12 optionally including B27 or Ad-DF+++ (advanced Dulbecco's modified Eagle's/F12 medium supplemented with GultaMax, 1 M HEPES)), an R-spondin family protein, a mitogenic growth factor (such as EGF), a BMP inhibitor (such as noggin), a TGF-beta inhibitor (such as A83-01), a p38 inhibitor (such as SB202190) and optionally nicotinamide and N-acetylcysteine.

In some embodiments the culture medium comprises advanced DMEM/F12 medium including B27, nicotinamide, N-acetylcysteine, noggin, R-spondin 1-4, EGF, Wnt (either WNT conditioned media (50%, produced using stably transfected L cells) or NGS Wnt), TGF-b type I receptor inhibitor A83-01 and P38 inhibitor SB202190.

The examples demonstrate that the claimed methods are particularly effective a creating intestinal organoids that have a mutational signature such as a colorectal cancer mutational signature. In this embodiment, the method for co-culturing genotoxic bacteria and intestinal organoids comprises culturing the organoid or organoid fragment in a culture medium comprising an R-spondin family protein, a mitogenic growth factor (such as EGF), a BMP inhibitor (such as noggin), a TGF-beta inhibitor. In some embodiments, the method for co-culturing genotoxic bacteria and intestinal organoids comprises culturing the organoid or organoid fragment in a culture medium comprising N-Acetylcysteine, TGFβ Receptor inhibitor A83-01; B27 supplement, EGF, Gastrin, Noggin, Nicotinamide, Rspondin-3, P38 inhibitor SB202190, MAPK inhibitor and NGS Wnt. In preferred embodiments, the method for co-culturing genotoxic bacteria and intestinal organoids comprises culturing the organoid or organoid fragment in a culture medium comprising Ad-DF++, N-Acetylcysteine (0.1-1 mM), TGFß Receptor inhibitor A83-01 (100-800 nM); B27 supplement (1×-5×), EGF (20-80 ng/ml), Gastrin (2-10 nM), Noggin (1-5% conditioned media from UPE or 50-250 ng/ml Recombinant Noggin (N)), Nicotinamide (2-20 mM), Rspondin 1-4 (100-500 ng/ml), SB202190 P38 MAPK inhibitor (2-20 μM) and NGS Wnt (0.1-2 nM).

### Extracellular Matrix and Synthetic Matrix

Epithelial stem cells are normally grown in culture with an exogenous extracellular matrix (ECM) that is known to support cell growth (e.g. see [12]). The culture medium suitable for culturing organoids and organoid fragments can comprise an ECM. The ECM is an exogenous ECM (meaning that it is in addition to any extracellular matrix proteins that are naturally secreted by the epithelial stem cell or population of epithelial stem cells when in contact with the expansion medium of the invention). Any suitable ECM may be used. Cells are preferably cultured in a microenvironment that mimics at least in part a cellular niche in which said cells naturally reside. A cellular niche is in part determined by the cells and by an ECM that is secreted by the cells in said niche. A cellular niche may be mimicked by culturing said cells in the presence of biomaterials or synthetic materials that provide interaction with cellular membrane proteins, such as integrins. An ECM as described herein is thus any biomaterial or synthetic material or combination thereof that mimics the in vivo cellular niche, e.g. by interacting with cellular membrane proteins, such as integrins.

In another embodiments, the ECM is in suspension, i.e. the cells are in contact with the ECM in a suspension system. In some embodiments, the ECM is in the suspension at a concentration of at least 1%, at least 2% or at least 3%. In some embodiments, the ECM is in the suspension at a concentration of from 1% to about 10% or from 1% to about 5%. The suspension method may have advantages for upscale methods.

One type of ECM is secreted by epithelial cells, endothelial cells, parietal endoderm like cells (e.g. Englebreth Holm Swarm Parietal Endoderm Like cells described in [13]) and connective tissue cells. This ECM comprises of a variety of polysaccharides, water, elastin, and glycoproteins, wherein the glycoproteins comprise collagen, entactin (nidogen), fibronectin, and laminin. Therefore, in some embodiments, the ECM for use in the methods of the invention comprises one or more of the components selected from the list: polysaccharides, elastin, and glycoproteins, e.g. wherein the glycoproteins comprise collagen, entactin (nidogen), fibronectin, and/or laminin. For example, in some embodiments, collagen is used as the ECM. Different types of ECM are known, comprising different compositions including different types of glycoproteins and/or different combination of glycoproteins.

The ECM can be provided by culturing ECM-producing cells, such as for example epithelial cells, endothelial cells, parietal endoderm like cells or fibroblast cells, in a receptacle, prior to the removal of these cells and the addition of isolated tissue fragments or isolated epithelial cells. Examples of extracellular matrix-producing cells are chondrocytes, producing mainly collagen and proteoglycans, fibroblast cells, producing mainly type IV collagen, laminin, interstitial procollagens, and fibronectin, and colonic myofibroblasts producing mainly collagens (type I, III, and V), chondroitin sulfate proteoglycan, hyaluronic acid, fibronectin, and tenascin-C. These are “naturally-produced ECMs”. Naturally-produced ECMs can be commercially provided. Examples of commercially available extracellular matrices include: extracellular matrix proteins (Invitrogen) and basement membrane preparations from Engelbreth-Holm-Swarm (EHS) mouse sarcoma cells (e.g. Cultrex® Basement Membrane Extract (Trevigen, Inc.) or Matrigel™ (BD Biosciences)).

Therefore, in some embodiments, is a naturally-produced ECM. In some embodiments the ECM is a laminin-containing ECM such as Matrigel™ (BD Biosciences). In some embodiments, the ECM is Matrigel™ (BD Biosciences), which comprises laminin, entactin, and collagen IV. In some embodiments, the ECM comprises laminin, entactin, collagen IV and heparin sulphate proteoglycan (e.g. Cultrex® Basement Membrane Extract Type 2 (Trevigen, Inc.)). In some embodiments, the ECM comprises at least one glycoprotein, such as collagen and/or laminin. A preferred ECM for use in a method of the invention comprises collagen and laminin. A further preferred ECM comprises laminin, entactin, and collagen IV. Mixtures of naturally-produced or synthetic ECM materials may be used, if desired.

In another embodiment, the ECM may be a synthetic ECM. For instance, a synthetic ECM, such as ProNectin (Sigma Z378666) may be used. In a further example, the ECM may be a plastic, e.g. a polyester, or a hydrogel. In some embodiments, a synthetic matrix may be coated with biomaterials, e.g. one or more glycoprotein, such as collagen or laminin.

In some embodiments, the first phase, the second phase and/or the third phase comprise culturing in the presence of an extracellular matrix. In preferred embodiments, the second phase and/or the third phase comprise culturing in the presence of an extracellular matrix. In some embodiments, the extracellular matrix is at least 50%, at least 60% or at least 70% Matrigel, optionally 50-100%, 50-80% Matrigel, optionally about 70% Matrigel. In preferred embodiments, the second phase and the third phase comprise culturing in the presence of an extracellular matrix, wherein the extracellular matrix is at least 70% Matrigel.

In some embodiments, the ECM is a three-dimensional matrix. In some embodiment, the cells are embedded in the ECM. In some embodiments, the cells are attached to an ECM. A culture medium of the invention may be diffused into a three-dimensional ECM. In other embodiments, the ECM is in suspension, i.e. the cells are in contact with the ECM in a suspension system. In some embodiments, the ECM is in the suspension at a concentration of at least 1%, at least 2% or at least 3%. In some embodiments, the ECM is in the suspension at a concentration of from 1% to about 10% or from 1% to about 5%.

In some embodiments, the culture methods of the invention comprise culturing epithelial stem cells in contact with an extracellular matrix. “In contact” means a physical or mechanical or chemical contact, which means that for separating said resulting organoid or population of epithelial cells from said matrix a force needs to be used. The culture medium and/or cells may be placed on, embedded in or mixed with the extracellular matrix or synthetic matrix.

In some embodiments, the culture medium is placed on top of the extracellular matrix or synthetic matrix. The culture medium can then be removed and replenished as and when required. In some embodiments, the culture medium is replenished every 1, 2, 3, 4, 5, 6 or 7 days. If components are “added” or “removed” from the media, then this can in some embodiments mean that the media itself is removed from the extracellular matrix or synthetic matrix and then a new media containing the “added” component or with the “removed” component excluded is placed on the extracellular matrix or synthetic matrix.

A three-dimensional matrix supports culturing of three-dimensional epithelial organoids. Therefore in some embodiments, the extracellular matrix or the synthetic matrix is a three-dimensional matrix.

In some embodiments, the medium further comprises an integrin agonist (e.g. as described in WO2020/234250). Specific examples of integrin agonists include anti-integrin antibodies, such as anti-b1 integrin antibodies (e.g. TS2/16, 12G10, 8A2, 15/7, HUTS-4, 8E3, N29 and 9EG7 antibodies). The integrin agonist may be used instead of or in addition to the extracellular matrix.

### Wnt Agonist

The culture medium suitable for culturing organoids and organoid fragments can comprise a Wnt agonist. The Wnt signalling pathway and small molecules which activate Wnt signalling are described in [14]. A Wnt agonist is defined herein as an agent that activates or enhances TCF/LEF-mediated transcription in a cell. Wnt agonists are therefore selected from true Wnt agonists that bind and activate the Wnt receptor complex including any and all of the Wnt family proteins, an inhibitor of intracellular β-catenin degradation, a GSK inhibitor (such as CHIR9901) and activators of TCF/LEF. The one or more Wnt agonist in the culture medium may be selected from a Wnt ligand from the Wnt family of secreted glycoproteins, an inhibitor of intracellular β-catenin degradation, a GSK-3 inhibitor, activators of TCF/LEF, an inhibitor of RNF43 or ZNRF3, and R-spondin family proteins. In some embodiments, the Wnt agonist in the culture medium comprises an R-spondin family protein and a GSK-3 inhibitor, and optionally further comprises a Wnt ligand from the Wnt family of secreted glycoproteins. One or more, for example, 2, 3, 4 or more Wnt agonists may be used in the culture medium. In one embodiment, the medium comprises an Lgr5 agonist, for example Rspondin, and additionally comprises a further Wnt agonist. In this context, the further Wnt agonist may, for example, be selected from the group consisting of Wnt-3a, a GSK-inhibitor (such as CHIR99021), Wnt-5, Wnt-6a Norrin, and NGS-Wnt. In one embodiment, the medium comprises Rspondin and additionally comprises a soluble Wnt ligand, such as Wnt3a or NGS-Wnt. Addition of a soluble Wnt ligand has been shown to be particularly advantageous for expansion of human epithelial stem cells (as described in WO2012/168930).

The R-spondin family protein (also referred to herein as “R-spondin”) may be selected from R-spondin 1, R-spondin 2, R-spondin 3, R-spondin 4 and analogs, fragments, variants and derivatives thereof. In this context, the fragment, variant or derivative is capable of preventing the action of the E3 ligases RNF43/ZNRF3 on the Wnt receptor complex. R-spondin 1, R-spondin 2, R-spondin 3 and R-spondin 4 (also referred to herein as “R-spondin 1-4”) are all characterized by two amino-terminal furin-like repeats, which are necessary and sufficient for Wnt signal potentiation, and a thrombospondin domain situated more towards the carboxyl terminus members [15]. Examples of R-spondin fragments, variants and derivatives suitable for use in the invention are known to the skilled person (e.g. see Example 2 of WO 2012/140274, which describes furin domain fragments which are capable of enhancing Wnt signalling and which are incorporated herein by reference). Examples of R-spondin family protein analogs include, for example, antibodies that interact with RNF43/ZNRF3/Lgr. Agonistic anti-Lgr5 antibodies that can enhance Wnt signalling are known in the art (e.g. see antibody 1D9 described in Example 3 of [16]).

Many GSK-3 inhibitors are known in the art (e.g. see [17]; and [18]) and are available commercially (e.g. see the list available from Santa Cruz Biotechnology here: https://www.scbt.com/scbt/browse/GSK-3-beta-Inhibitors//N-x6oud). Any of these GSK-3 inhibitors are suitable for use in the context of the invention and the skilled person would be able to determine a suitable concentration using IC50 values.

CHIR-99021 (CAS: 252917-06-9; 6-[[2-[[4-(2,4-Dichlorophenyl)-5-(5-methyl-1H-imidazol-2-yl)-2-pyrimidinyl]amino]ethyl]amino]-3-pyridinecarbonitrile; CT99021) is a potent and selective inhibitor of GSK-3. Other aminopyrimidine inhibitors with an IC50 value of 0.6 nM to 7 nM include CHIR98014 (Axon, Cat 1126), CHIR98023, CHIR99021 (see above), TWS119 (Tocris, Cat 3835). Therefore, in some embodiments, the GSK-3 inhibitor is an aminopyrimidine inhibitor, optionally selected from CHIR98014, CHIR98023, CHIR99021 or TWS119. In some embodiments the GSK-3 inhibitor is CHIR-99021.

The Wnt ligand from the Wnt family of secreted glycoproteins may be selected from Wnt-I/Int-1, Wnt-2/Irp (InM-related Protein), Wnt-2b/13, Wnt-3/Int-4, Wnt-3a (R&D systems), Wnt-4, Wnt-5a, Wnt-5b, Wnt-6 (see [19]), Wnt-7a (R&D systems), Wnt-7b, Wnt-8a/8d, Wnt-8b, Wnt-9a/14, Wnt-9b/14b/15, Wnt-10a, Wnt-10b/12, WnM I, and Wnt-16. An overview of human Wnt proteins is provided in [20]. In some embodiments, the Wnt ligand is Wnt-3a, Wnt-5 or Wnt-6a, or optionally is Wnt-3a. Addition of a soluble Wnt ligand has been shown to be particularly advantageous for expansion of human organoids and organoid fragments (e.g. as described in [21]).

In some embodiments, the Wnt agonist in the culture medium is a Wnt surrogate. Wnt surrogate is a water-soluble Wnt agonist engineered by linking antagonistic Fzd and Lrp5/6-binding modules into a single polypeptide chain, thus forcing receptor heterodimerisation while blocking endogenous Wnt binding. Wnt surrogate supports the growth of a broad range of cultures. Furthermore, Wnt surrogate is a non-lipidated Wnt agonists that can be produced in serum free medium, kept frozen and circumvent the differences in activity of Wnt-conditioned media produced by different laboratories [22]. In some embodiments, the Wnt surrogate is next-generation surrogate Wnt (NGS-Wnt), for example as described in [23]. NGS-Wnt may be provided at a concentration of about 0.1 nM to about 0.5 nM. In some embodiments, the medium comprises NGS-Wnt at a concentration of about 0.5 nM. In some embodiments, the medium comprises NGS-Wnt at a concentration of about 0.1 nM.

### Mitogenic Growth Factors

The culture medium suitable for culturing organoids and organoid fragments can comprise a mitogenic growth factor. Mitogenic growth factors typically induce cell division via the mitogen-activated protein kinase signalling pathway. Many receptor tyrosine kinase ligands are mitogenic growth factors. In some embodiments, the mitogenic growth factor can bind to a receptor tyrosine kinase. In some embodiments, the mitogenic growth factor can bind to more than one receptor tyrosine kinase. In some embodiments, the one or more mitogenic growth factor binds to a receptor tyrosine kinase such as EGFR, an FGFR or HGFR, optionally wherein the one or more mitogenic growth factors are selected from EGF, FGF and HGF.

In some embodiments, the mitogenic growth factor binds to EGFR, HER1, HER2, HER3 or HER4. In some embodiments, the mitogenic growth factor binds to EGFR. In some embodiments, a HER2-4 ligand is included in the culture medium in addition to an EGFR ligand. For example, in some embodiments, neuregulin is included in the culture medium in addition to EGF. Neuregulin has been shown to be advantageous for culture of lung and breast tissue (e.g. see [24], and [25]). In some embodiments, the one or more mitogenic growth factor in the culture medium is EGF. Any suitable EGF may be used, for example, EGF obtained from Peprotech.

FGFs stimulate cells by interacting with cell surface tyrosine kinase receptors (FGFR). Four closely related receptors (FGFR1-FGFR4) have been identified. Therefore, in some embodiments, the mitogenic growth factor binds to an FGF receptor family member. FGF receptor family members include (but are not limited to) FGFR1, FGFR2, FGFR3 or FGFR4. FGFR1-FGFR3 genes have been shown to encode multiple isoforms, and these isoforms can be critical in determining ligand specificity. There are several FGFs that bind to the FGF receptor family members, including (but not limited to) FGF2, FGF4, FGF7 and FGF10. These are commercially available. Therefore, in some embodiments, the mitogenic growth factor is an FGF. In some embodiments, the FGF is selected from FGF2, FGF4, FGF7 and FGF10. In preferred embodiments, the FGF is FGF2 and/or FGF10. In a most preferred embodiment, the FGF is FGF2 and FGF10.

Hepatocyte growth factor/scatter factor (HGF/SF) is a morphogenic factor that regulates cell growth, cell motility, and morphogenesis by activating a tyrosine kinase signalling cascade after binding to the proto-oncogenic HGFR. The HGFR is also known as the c-Met receptor. HGF has been shown to be useful in epithelial stem cell culture. Therefore, in some embodiments the mitogenic growth factor binds HGFR. In some embodiments, the mitogenic growth factor is HGF. Any suitable HGF may be used, for example, HGF obtained from Peprotech.

In some embodiments, more than one mitogenic growth factor is included in the culture medium, e.g. two or three mitogenic growth factors. For example, in some embodiments, the one or more mitogenic growth factors in the culture medium are EGF and FGF. In some embodiments, the one or more mitogenic growth factors in the culture medium are EGF, FGF2 and FGF10. In some embodiments, the one or more mitogenic growth factors in the culture medium are EGF, optionally at a final concentration of about 50 ng/ml, FGF2, optionally at a final concentration of about 5 ng/ml, and FGF10, optionally at a final concentration of about 10 ng/ml.

In some embodiments hepatocyte growth factor (HGF) is also present in the presence or absence of EGF and/or FGF.

### Bmp Inhibitor

The culture medium suitable for culturing organoids and organoid fragments can comprises a BMP inhibitor. A BMP inhibitor is defined as an agent that binds to a BMP molecule to form a complex wherein the BMP activity is neutralized, for example by preventing or inhibiting the binding of the BMP molecule to a BMP receptor. Alternatively, said inhibitor is an agent that acts as an antagonist or reverse agonist. This type of inhibitor binds with a BMP receptor and prevents binding of a BMP to said receptor. An example of a latter agent is an antibody that binds a BMP receptor and prevents binding of BMP to the antibody-bound receptor.

A BMP inhibitor may be added to the media in an amount effective to inhibit a BMP-dependent activity in a cell to at most 90%, more preferred at most 80%, more preferred at most 70%, more preferred at most 50%, more preferred at most 30%, more preferred at most 10%, more preferred 0%, relative to a level of a BMP activity in the absence of said inhibitor, as assessed in the same cell type. As is known to a skilled person, a BMP activity can be determined by measuring the transcriptional activity of BMP, for example as exemplified in Zilberberg et al., 2007. BMC Cell Biol. 8:41.

Several classes of natural BMP-binding proteins are known, including noggin (Peprotech), Chordin and chordin-like proteins (R&D systems) comprising chordin domains, Follistatin and follistatin-related proteins (R&D systems) comprising a follistatin domain, DAN and DAN-like proteins (R&D systems) comprising a DAN cysteine-knot domain, sclerostin/SOST (R&D systems), decorin (R&D systems), and alpha-2 macroglobulin (R&D systems).

Therefore, in some embodiments, the BMP inhibitor is selected from noggin, DAN, and DAN-like proteins including Cerberus and Gremlin (R&D systems). These diffusible proteins are able to bind a BMP ligand with varying degrees of affinity and inhibit their access to signalling receptors. The addition of any of these BMP inhibitors to the basal culture medium prevents the loss of stem cells. A preferred BMP inhibitor is noggin.

### TGF-Beta Inhibitor

The culture medium suitable for culturing organoids and organoid fragments can comprises a TGF-beta inhibitor. The presence of a TGF-beta inhibitor in the expansion media is particularly advantageous for increasing human organoid formation efficiency. A TGF-beta inhibitor is any agent that reduces the activity of the TGF-beta signalling pathway, also referred to herein as the ALK4, ALK5 or ALK7 signalling pathway. A TGF-beta inhibitor according to the present invention may be a protein, peptide, small-molecule, small-interfering RNA, antisense oligonucleotide, aptamer or antibody. The inhibitor may be naturally occurring or synthetic.

In some embodiments the TGF-beta inhibitor is a small molecule inhibitor, such as A83-01. A83-01 is a commercially available selective inhibitor of ALK4, ALK5 and ALK7 (Tocris cat. no. 2939). It is described in the catalog as a potent inhibitor of TGF-β type I receptor ALK5 kinase, type I activin/nodal receptor ALK4 and type I nodal receptor ALK7 (IC50 values are 12, 45 and 7.5 nM respectively), which blocks phosphorylation of Smad2, and which only weakly inhibits ALK-1,-2,-3,-6 and MAPK activity. Other commercially available inhibitors with similar properties include, but are not limited to A77-01, LY2157299, LY2109761, LY3200882, GW788388, Pirfenidone, RepSox, SB431542, SB505124, SB525334, LY364947, SD-208 and Vactosertib. The IC50 values for these inhibitors are known in the art and the skilled person would be able to select a suitable inhibitor at suitable concentration based on the teaching provided in the examples of this application.

### Nicotinamide

In some embodiments, the culture medium suitable for organoids and organoid fragments further comprises nicotinamide. Nicotinamide is an amide derivative of vitamin B3, a poly (ADP-ribose) polymerase (PARP) inhibitor, and represents the primary precursor of NAD+. It is available commercially (e.g. from Stemcell Technologies Cat. 07154).

### Prostaglandin Pathway Activator

In some embodiments, the culture medium suitable for organoids and organoid fragments further comprises a prostaglandin pathway activator. The prostaglandin pathway activator may be any one or more of the compounds selected from the list comprising: phospholipids, arachidonic acid (AA), prostaglandin E2 (PGE2), prostaglandin G2 (PGG2), prostaglandin F2 (PGF2), prostaglandin H2 (PGH2), prostaglandin D2 (PGD2). In some embodiments, the activator of the prostaglandin signalling pathway is PGE2 and/or AA. In some embodiments, the activator of the prostaglandin signalling pathway is PGE2.

### Camp Activator

In some embodiments, the culture medium suitable for organoids and organoid fragments further comprises a CAMP pathway activator. The CAMP pathway activator may be any suitable activator which increases the levels of CAMP in a cell. In some embodiments, the CAMP pathway activator is an adenylyl cyclase activator or a CAMP analog. Examples of suitable adenylyl cyclase activators include forskolin, a forskolin analog and cholera toxin. Examples of forskolin analogs are known in the art and include NKH477 (e.g. catalogue no. Tocris 1603). Examples of CAMP analogs are also known in the art, and include for example, 8-bromo-cAMP. 8-bromo-CAMP is a cell-permeable cAMP analog having greater resistance to hydrolysis by phosphodiesterases than CAMP. In some embodiments, the CAMP pathway activator is therefore selected from forskolin, cholera toxin, NKH477 and 8-bromo-cAMP. In some embodiments, the CAMP pathway activator is forskolin. In some embodiments, the CAMP pathway activator is not cholera toxin.

### Additional Components

Basal media for cell culture typically contain a large number of ingredients, which are necessary to support maintenance of the cultured cells. Suitable combinations of ingredients can readily be formulated by the skilled person, taking into account the following disclosure. A basal medium for use in the invention will generally comprises a nutrient solution comprising standard cell culture ingredients, such as amino acids, vitamins, lipid supplements, inorganic salts, a carbon energy source, and a buffer, as described in more detail in the literature and below. In some embodiments, the culture medium is further supplemented with one or more standard cell culture ingredient, for example selected from amino acids, vitamins, lipid supplements, inorganic salts, a carbon energy source, and a buffer. Suitable basal media will be known to the skilled person and are available commercially, e.g. non-limiting examples include Dulbecco's Modified Eagle Media (DMEM), Advanced-DMEM, Minimal Essential Medium (MEM), Knockout-DMEM (KO-DMEM), Glasgow Minimal Essential Medium (G-MEM), Basal Medium Eagle (BME), DMEM/Ham's F12, Advanced DMEM/Ham's F12, Iscove's Modified Dulbecco's Media and Minimal Essential Media (MEM), Ham's F-10, Ham's F-12, Medium 199, and RPMI 1640 Media. For example, the basal medium may be Advanced-DMEM, preferably supplemented with glutamax, penicillin/streptomycin and HEPES.

The culture medium suitable for organoids and organoid fragments may be supplemented with one or more of the compounds selected from the group consisting of gastrin, B27, N-acetylcystein and N2. Thus in some embodiments the culture medium described above further comprises one or more components selected from the group consisting of: gastrin, B27, N2 and N-Acetylcysteine. B27 (Invitrogen), N-Acetylcysteine (Sigma) and N2 (Invitrogen), Gastrin (Sigma) are believed to control proliferation of the cells and assist with DNA stability. In some embodiments, the culture medium further comprises B27 and N-acetylcystein.

In some embodiments the culture medium further comprises a ROCK inhibitor (Rho-Kinase inhibitor). A ROCK inhibitor is particularly useful for attachment of cells when establishing new cultures and/or when splitting (“passaging”) cells. Suitable ROCK inhibitors are known in the art and available commercially (including but not limited to GSK 269962, GSK 429286, H 1152 dihydrochloride, Glycyl-H 1152 dihydrochloride, SR 3677 dihydrochloride, SB 772077B dihydrochloride and Y-27632 dihydrochloride, all available from Tocris). In some embodiments the medium is supplemented with 5 μM to 20 μM or 8 μM to 15 μM ROCK inhibitor, optionally about 10 μM ROCK inhibitor.

It is preferred that the culture medium does not comprise an undefined component (such as fetal bovine serum or fetal calf serum or feeder cells). Various different serum replacement formulations are commercially available and are known to the skilled person. Where a serum replacement is used, it may be used at between about 1% and about 30% by volume of the medium, according to conventional techniques. In some embodiments, the culture medium is serum free and/or feeder free.

A preferred cell culture medium is a defined synthetic medium that is buffered at a pH of 7.4 (preferably with a pH 7.2-7.6 or at least 7.2 and not higher than 7.6) with a carbonate-based buffer, while the cells are cultured in an atmosphere comprising between 5% and 10% CO2, or at least 5% and not more than 10% CO2, preferably 5% CO2.

### Uses of the Co-Culture Organoid Model

The invention provides method for drug screening, target validation, target discovery, toxicology or a toxicology screen using the co-culture methods of the invention. For example, the invention provides a method for testing the effect of a candidate compound, wherein the method comprises:
exposing the co-culture of genotoxic bacteria and organoids from methods of the invention to one of a library of candidate compounds;
evaluating the organoids in the co-culture for any effects (e.g. compared to a control which has not been exposed to a candidate compound),
identifying the candidate molecule that causes said effects as a potential drug.
   The genotoxic bacteria can be precultured with one of a library of candidate compounds and then introduced into the co-culture methods. Alternatively, one of a library of candidate compounds can be introduced in the first phase, the second phase and/or the third phase of the co-culture method. In some embodiments, the one of a library of candidate compounds is introduced in one phase, two phases or all three phases. These methods can be used to test libraries of chemicals, antibodies, natural products (e.g. plant extracts or microbial compounds), etc for suitability for use as drugs, cosmetics and/or preventative medicines.

The invention also provides the use of an organoid with a mutational signature produced by the methods of the invention in drug screening, target validation, target discovery, toxicology or a toxicology screen. In some embodiments, the organoids are used for modelling host-pathogen interaction and/or for identifying compounds suitable for treating bacterial infection and/or cancer.

For example, the cells are preferably exposed to multiple concentrations of a test agent for a certain period of time. At the end of the exposure period, the cultures are evaluated. The organoid can also be used to identify drugs that specifically target epithelial carcinoma cells. It will be understood by the skilled person that the organoids of the invention would be widely applicable as drug screening tools for infectious, inflammatory and neoplastic pathologies. In some embodiments, the invention provides the use of an organoid in drug screening, target validation, target discovery, toxicology, toxicology screens or an ex vivo cell/organ model. In some embodiments, the invention provides the use of an organoid in an ex vivo method to predict a clinical outcome. In some embodiments, the organoids of the invention could be used for screening for cancer drugs.

In some embodiments, the organoids of the invention can be used to test libraries of chemicals, antibodies, natural products (e.g. plant extracts or microbial compounds), etc for suitability for use as drugs, cosmetics and/or preventative medicines.

In some embodiments, the invention provides a method for testing the effect of a candidate compound, wherein the method comprises:
exposing the organoid with a mutational signature to one or a library of candidate compounds;
   evaluating said expanded organoids for any effects (e.g. compared to a control which has not been exposed to a candidate compound),
   identifying the candidate molecule that causes said effects as a potential drug.

In some embodiments, the method for testing the effect of a candidate compound comprises exposing the organoid with a mutational signature to radiation in the presence or absence of a candidate compound. In some embodiments, an evaluated effect in the method for testing the effect of a candidate compound is selected from the list comprising: a reduction in, or loss of, proliferation, a morphological change, cell death or a change in gene or protein expression. A library of candidate molecules comprises more than one candidate molecule.

In some embodiments, the invention provides a method comprising:
exposing the organoid with a mutational signature or a population of cells derived from the organoid with a mutational signature to a treatment, such as radiation, and/or to one or a library of candidate molecules;
evaluating said organoid or population of cells for any effects of a candidate molecule; and
correlating said effect with a feature of the organoid, for example the presence of one or more genetic mutations, such as mutations in the EGFR signalling pathway, including PIK3CA, KRAS, HRAS or BRAF.

In some embodiments, the invention provides a method comprising:
exposing the organoid or a population of cells derived from the organoid to a treatment, such as radiation, and/or to one or a library of candidate molecules;
evaluating said organoid or population of cells for any effects of a candidate molecule;
comparing said effect with standard values and/or previous observations; and optionally
predicting clinical outcome and/or selecting a personalised medicine.

### Definitions

As used herein, the verb “to comprise” and its conjugations is used in its non-limiting sense to mean that items following the word are included, but items not specifically mentioned are not excluded. In addition the verb “to consist” may be replaced, if necessary, by “to consist essentially of′ meaning that a product as defined herein may comprise additional component(s) than the ones specifically identified, said additional component(s) not altering the unique characteristic of the invention. In addition a method as defined herein may comprise additional step(s) than the ones specifically identified, said additional step(s) not altering the unique characteristic of the invention. In addition, reference to an element by the indefinite article “a” or “an” does not exclude the possibility that more than one of the element is present, unless the context clearly requires that there be one and only one of the elements. The indefinite article “a” or “an” thus usually means “at least one”.

As used herein, the term “about” or “approximately” means that the value presented can be varied by +/−10%. The value can also be read as the exact value and so the term “about” can be omitted. For example, the term “about 100” encompasses 90-110 and also 100.

All patent and literature references cited in the present specification are hereby incorporated by reference in their entirety.

Any reference to a method for treatment comprising administering an agent to a patient, also covers that agent for use in said method for treatment, as well as the use of the agent in said method for treatment, and the use of the agent in the manufacture of a medicament.

The following examples are offered for illustrative purposes only, and are not intended to limit the scope of the present invention in any way.

The practice of the present invention will employ, unless otherwise indicated, conventional methods of chemistry, biochemistry, molecular biology, immunology and pharmacology, within the skill of the art. Such techniques are explained fully in the literature. See, e.g., references [26-32], etc.

### EMBODIMENTS

The invention provides the following numbered embodiments:

1. A method for co-culturing genotoxic bacteria and organoids, wherein the method comprises at least two phases selected from:
a first phase comprising culturing organoids or organoid fragments with genotoxic bacteria;
a second phase comprising culturing the organoids or organoids fragments with genotoxic bacteria, wherein the bacterial growth is restricted with antibiotics,
a third phase comprising killing the genotoxic bacteria and enabling the further growth of the organoids or organoid fragments.

2. The method of embodiment 1, wherein the method produces an organoid or an organoid fragment that has a mutational signature.

3. The method according to any preceding embodiment, wherein the genotoxic bacteria is introduced in the first phase and prevails in the system for further culture in the second phase.

4. The method according to any preceding embodiment, wherein the organoid fragments are obtained by shearing one or more organoids.

5. The method according to any preceding embodiment, wherein during the second phase at least 60%, at least 70%, at least 80% or at least 90% of the organoid fragments form one or more organoids.

6. The method according to any preceding embodiment, wherein the method comprises:
(a) the first and the second phase only;
(b) the first and the third phase only;
(c) the second and the third phase only; or
(d) the first, the second phase and the third phase.

7. The method according to any preceding embodiment, wherein the phases in the method are repeated at least twice, at least three times, at least four times or at least five times, optionally wherein the phases in the method are repeated at least five times.

8. The method according to any preceding embodiment, wherein in the genotoxic bacteria is in exponential growth phase when it is incorporated into the co-culture.

9. The method according to any preceding embodiment, wherein the genotoxic bacteria has a multiplicity of infection of between 0.1 and 800, between 10 and 100, between 50 and 100, or about 100 when it is incorporated into the co-culture.

10. The method according to any preceding embodiment, wherein the growth of the rate of the genotoxic bacteria in the second phase is lower than the growth of the rate of the genotoxic bacteria in the first phase, optionally wherein the growth rate of the genotoxic bacteria in the second phase is reduced by at least 10%, 20%, 30%, 40% or 50% compared to the growth rate of the genotoxic bacteria in the first phase.

11. The method according to any preceding embodiment, wherein the growth of the rate of the genotoxic bacteria in the second phase is restricted using an antibiotic.

12. The method according to any preceding embodiment, wherein the organoid fragment or organoid is derived from gastrointestinal tract, stomach, pancreas, lung, mouth, nasopharynx, throat, hypopharynx, larynx, trachea, skin, vaginal, and/or esophagus tissue, optionally wherein the organoid fragment or organoid is derived from the gastrointestinal tract.

13. The method according to any preceding embodiment, wherein the first phase occurs for between 15 mins to 5 hr.

14. The method according to any preceding embodiment, wherein the second phase occurs for between 2 days to 3 days

15. The method according to any preceding embodiment, wherein the third phase occurs for between 1 day to 20 days.

16. The method according to any preceding embodiment, wherein the genotoxic bacteria is a colibactin-producing bacteria.

17. The method according to embodiment 16, wherein the colibactin-producing bacteria is from the family Enterobacteriaceae.

18. The method according to embodiment 17, wherein the colibactin-producing bacteria is from the species E coli.

19. The method according to embodiment 18, wherein the colibactin-producing bacteria is pks+E. coli.

20. The method of any preceding embodiment, wherein the first phase occurs in suspension culture.

21. The method of any preceding embodiment, wherein the second and/or the third phase comprises culturing in the presence of an extracellular matrix, optionally wherein the extracellular matrix is Matrigel or Basement Membrane Extract.

22. The method according to any preceding embodiment, wherein the method produces an organoid that has a mutational signature.

23. The method according to embodiment 22, wherein the method produces an organoid that has a cancer mutational signature.

24. A method of producing an organoid with a mutational signature, wherein the method comprises at least two phases selected from:
a first phase comprising culturing organoids or organoid fragments with genotoxic bacteria;
a second phase comprising culturing the organoids or organoids fragments with genotoxic bacteria, wherein the bacterial growth is restricted with antibiotics,
a third phase comprising killing the genotoxic bacteria and enabling the further growth of the organoids or organoid fragments, wherein an organoid or an organoid fragment that has a mutational signature is produced.

25. The method of embodiment 24, wherein the mutational signature is a cancer mutational signature.

26. The method of embodiment 24 or 25, further comprising a step of detecting DNA damage and/or determining the mutational signature.

27. The method of any one of embodiments 24-26, further comprising a step of treating the organoid produced by the method with one or more test compounds to identify a compound suitable for treating bacterial infection and/or cancer.

28. The method of any one of embodiments 22-27, wherein the organoid produced by the method is a medium or high throughput platform, suitable for modelling host (organoid)-pathogen (bacterial) interaction.

29. A method for testing the effect of a candidate compound, wherein the method comprises:
exposing the co-culture of genotoxic bacteria and organoids from the method according to any preceding embodiment to one of a library of candidate compounds;
evaluating the organoids in the co-culture for any effects,
identifying the candidate molecule that causes said effects as a potential drug.

30. The method according to embodiment 29, wherein the genotoxic bacteria is precultured with one of a library of candidate compounds and then introduced into the co-culture methods of any preceding embodiment.

31. An organoid with a mutational signature produced by the method of any one of the preceding embodiments.

32. The organoid of embodiment 31, wherein the organoid is a gastrointestinal organoid with a colorectal cancer mutational signature.

33. Use of the organoid of embodiment 31 for modelling host-pathogen interaction and/or for identifying compounds suitable for treating bacterial infection and/or cancer.

### EXAMPLES

### Example 1—Methods for Co-Culturing Genotoxic Bacteria and Organoids Involving Three Phases

In this example, intestinal organoids were co-cultured with colibactin-producing pks+E. coli in order to model aspects of cancer initiation and progression. The co-culture strategy scheme tested consists of three phases as set out in FIG. 1A:
The first phase (Co-culture I)—a phase of ‘acute’ DNA damage during which the bacteria and organoid fragments interact in suspension and the growth of the bacteria is not restricted;
The second phase (Co-culture II)—a phase of ‘sustained’ DNA damage during which the organoid fragments and bacteria are cultured in hydrogels and bacteria growth is controlled by the addition of low concentrations of antibiotics;
The third phase (recovery phase)—a phase during which the bacteria are killed using an antibiotic and the organoids or organoid fragments continue growing in hydrogels.

### The First Phase

Processing of colon tissue resections for the establishment of organoid cultures was carried out according to the methods described in Sato et al. [33]. Organoids derived from a healthy donor were grown in 70% Matrigel for 2-4 days, sheared with a glass pipette and incubated with bacteria (MOI 0 [Bact]1, MOI 10 [Bact]2, MOI 50 [Bact]3, MOI 100 [Bact]4, MOI 500 [Bact]5 and MOI 1000 [Bact]6) in exponential growth phase (OD600>0.4 and <1) for 3-4 h in suspension in 0.5 medium (composition below) supplemented with 10 μM of Rhoki and in the absence of antibiotics. MOI (Multiplicity of infection) refers to the ratio of bacteria to organoid cell at the beginning of co-culture I. Organoids exposed to MOI 500 and MOI 1000 did not survive.

The composition of CSM media is Ad-DF++ (advanced Dulbecco's modified Eagle's/F12 medium supplemented with GultaMax, 1 M HEPES) containing 0.25 mM N-Acetylcysteine, 500 nM TGFβ Receptor inhibitor A83-01; 1× B27 supplement, 50 ng/ml EGF, 5 nM Gastrin, 2% Noggin conditioned media from UPE or 100 ng/ml Recombinant Noggin (N), 10 mM Nicotinamide, 250 ng/ml Rspondin-3, 10 μM SB202190 P38 MAPK inhibitor and 0.5 nM NGS Wnt.

FIG. 1B shows organoid fragments after 4 h of co-culture with E. coli (green) MOI 100 in suspension 0.5 CSM medium supplemented with 10 μM of Rhoki, 10% Matrigel and in the absence of antibiotics. Bacteria were stained with CFSE for 30 min in PBS prior to co-culture. Organoid cell nuclei (blue) were stained with DAPI. Phalloidin (orange) stained the apical side of the organoid fragments. The addition of 10% Matrigel in this experiment enabled the immobilization of bacteria near the organoid cells and subsequent visualization.

### The Second Phase

After co-culture I, organoids were washed with AdDMEM medium and plated in 70% Matrigel. After Matrigel solidification, organoids were cultured in 0.5 CSM medium supplemented with 50 μg/ml of gentamicin for 2 days.

### The Third Phase

The organoids were then cultured in 0.5 CSM containing 50 μg/ml Primocin and the organoids were left to recover for 2-5 days. The organoids that survived following co-culture with colibactin producing bacteria as shown in FIG. 1D.

The co-culture of organoid fragments with pks+E coli resulted in DNA damage as shown by the increase in the levels of yH2AX (which is a measure of the extent of DNA damage) in live single cells, as analyzed by flow cytometry. FIG. 1C shows a distribution of the organoid single cells derived from the co-culture as a function of yH2AX levels and at different time points, as obtained by flow cytometry. In this experiment the first phase lasted 3 h, the second phase for 2 days and the third phase was 3 days. yH2AX levels in live single cells were analyzed after 3 h of co-culture in suspension (the first phase), after 16 h of co-culture in 70% Matrigel in the presence of 50 μg/ml gentamicin (the second phase) and after 3 days of recovery in the presence of Primocin (the third phase). [Bact]1=MOI 1, [Bact]2=MOI 10, [Bact]3=MOI 100, [Bact]4=MOI 500. DNA damage was detected in a dose-dependent manner after the first phase. After 16 h of co-culture II, DNA damage was only detected in MOI 100. DNA damage was not detected following recovery.

### Example 2: DNA Damage Detected in Co-Culture of Genotoxic Bacteria and Organoids is Dependent of Colibactin Production

The co-culture scheme used in this example is shown in FIG. 2A. The co-culture strategy scheme tested consists of two phases, the first phase and the third phase as outlined above.

This is the first phase involved shearing organoid fragments cultured for 3 days with a glass pipette. Then, they were incubated with bacteria (No bacteria control=MOI 0; colibactin-producing and colibactin deficient (ΔClbP)=MOI 500) in exponential growth phase (OD600>0.4 and <1) for 4 h in suspension in 0.5 CSM medium supplemented with 10 μM of Rhoki and in the absence of antibiotics.

Organoid fragments were then washed with ADMEM and, for the recovery phase, cultured in suspension in 0.5 CSM medium supplemented with 10 μM Rhoki and 100 μg/ml gentamicin (the third phase). After 4 h of this recovery phase, organoids were washed and processed for staining (immunofluorescence (IF)). FIGS. 2B, C and D demonstrate that colibactin producing bacteria induced higher levels of DNA damage (levels of yH2AX) than colibactin-deficient (ΔClbP) bacteria in organoid fragments.

### Example 3: Methods for Co-Culturing Genotoxic Bacteria and Organoids Involving Two Phases

The co-culture scheme used in this example is shown in FIG. 3A. The co-culture strategy scheme tested consists of two phases, the first phase and the second phase as outlined above.

This is the first phase involved shearing organoid fragments cultured for 4 days with a glass pipette. Then, they were incubated with bacteria. No bacteria control=MOI 0; colibactin-producing (WT:KanR) and colibactin deficient (ΔClbP:KanR) bacteria at MOI 50, MOI 100, MOI 200 and WT MOI 300 in exponential growth phase (OD600>0.4 and <1) for 3 h in suspension in 0.5 CSM medium supplemented with 10 μM of Rhoki and in the absence of antibiotics.

The organoids and organoid fragments were washed with AdDMEM medium and plated in 70% Matrigel for 12 h in medium containing 50 μg/ml gentamicin. The organoids were washed and processed for immunofluorescence staining. FIG. 3B demonstrates that the organoids or organoid fragments exposed to bacterial MOI 50-300 survive to the acute DNA damage phase. FIGS. 3C and D show that organoid fragments exposed to colibactin-producing bacteria have enhanced yH2AX nuclear levels, a proxi for DNA damage, than organoid fragments exposed to colibactin-deficient bacteria and therefore demonstrates that colibactin producing bacteria induced higher levels of DNA damage (levels of yH2AX) than colibactin-deficient (ΔClbP) bacteria in organoid fragments.

### REFERENCES

[1] Faïs et al. (2018) Toxins, 10, 151; doi:10.3390/toxins10040151
[2] Pleguezuelos-Manzano, et al. (2020) Nature 580, 269-273
[3] Nougayrède et al. (2006) Science 313, 848
[4] Olive and Banath (2006) Nature Protocols volume 1, pages 23-29
[5] Ivashkevich et al (2012) Cancer Lett. 327 (1-2): 123-133. doi:10.1016/j.canlet.2011.12.025.
[6] Co et al. (2019) Cell Reports 26, 2509-2520
[7] Deris et al. (2013) Science, 342 (6162): 1237435.
[8] Martin et al. (2016) Bacterial Genotoxins as the Interphase Between DNA Damage and Immune Response. In: Microbial Toxins. Toxinology. Springer, Dordrecht. https://doi.org/10.1007/978-94-007-6725-6_14-1
[9] Clevers, Cell 165 (7) 1586-1597, (2016)
[10] Komiyama et al. (2021) Nature scientific reports 11:10589
[11] Zhang et al. (2021) Front. Oncol., 14 Jan. 2021|https://doi.org/10.3389/fonc.2020.604531
[12] M van de Wetering et al. (2015) Cell.; 161 (4): 933-45
[13] Hayashi et al. (2004) Matrix Biology 23:47 62
[14] Nusse and Clevers (2017) Cell 169 (6): 985-999
[15] Lau et al. Genome Biol. 2012; 13 (3): 242 (2012
[16] WO 2012/140274
[17] Greengard, P., and Meijer, L. (2004) Structural basis for the synthesis of indirubins as potent and selective inhibitors of glycogen synthase kinase-3 and cyclin-dependent kinases. J Med Chem 47:935-946
[18] Thomas Kramer, Boris Schmidt, and Fabio Lo Monte, “Small-Molecule Inhibitors of GSK-3: Structural Insights and Their Application to Alzheimer's Disease Models,” International Journal of Alzheimer's Disease, vol. 2012, Article ID 381029, 32 pages, 2012. https://doi.org/10.1155/2012/381029
[19] Kirikoshi H et al 2001 Biochem Biophys Res Com 283 798-805
[20] “THE WNT FAMILY OF SECRETED PROTEINS”, R&D Systems Catalog, 2004
[21] WO2012/168930
[22] Janda C Y, et al. (2017) Nature; 545 (7653):234-237
[23] Miao, Y. et al. (2020) Cell Stem Cell. 27 (5), 840-851
[24] WO2016/083613
[25] WO2016/083612
[26] Gennaro (2000) Remington: The Science and Practice of Pharmacy. 20th edition, ISBN: 0683306472.
[27] Molecular Biology Techniques: An Intensive Laboratory Course, (Ream et al., eds., 1998, Academic Press).
[28] Methods In Enzymology (S. Colowick and N. Kaplan, eds., Academic Press, Inc.)
[29] Handbook of Experimental Immunology, Vols. I-IV (D. M. Weir and C. C. Blackwell, eds, 1986, Blackwell Scientific Publications)
[30] Sambrook et al. (2001) Molecular Cloning: A Laboratory Manual, 3rd edition (Cold Spring Harbor Laboratory Press).
[31] Ausubel et al. (eds) (2002) Short protocols in molecular biology, 5th edition (Current Protocols).
[32] PCR (Introduction to Biotechniques Series), 2nd ed. (Newton & Graham eds., 1997, Springer Verlag)
[33] Sato et al., (2011 Gastroenterology, 2011, 141 (5): 1762-1772.

1. A method for co-culturing genotoxic bacteria and organoids, wherein the method comprises:
a first phase comprising culturing organoids or organoid fragments with genotoxic bacteria;
a second phase comprising culturing the organoids or organoid fragments with genotoxic bacteria, wherein the bacterial growth is restricted with antibiotics, and
a third phase comprising killing the genotoxic bacteria and enabling the further growth of the organoids or organoid fragments.

2. A method for co-culturing genotoxic bacteria and organoids, wherein the method comprises at least two phases selected from:
a first phase comprising culturing organoids with genotoxic bacteria;
a second phase comprising culturing the organoids with genotoxic bacteria, wherein the bacterial growth is restricted with antibiotics, and
a third phase comprising killing the genotoxic bacteria and enabling the further growth of the organoids or organoid fragments; and
wherein the method does not involve injecting the genotoxic bacteria into the lumen of the organoid.

3. A method for co-culturing genotoxic bacteria and organoid fragments, wherein the method comprises at least two phases selected from:
a first phase comprising culturing organoid fragments with genotoxic bacteria;
a second phase comprising culturing the organoid fragments with genotoxic bacteria, wherein the bacterial growth is restricted with antibiotics, and
a third phase comprising killing the genotoxic bacteria and enabling the further growth of the organoid fragments.

4. The method of claim 1 or claim 3, wherein the organoid fragments are obtained by shearing one or more organoids.

5. The method according to any one of claim 1, 3 or 4, wherein during the second phase at least 60%, at least 70%, at least 80% or at least 90% of the organoid fragments form one or more organoids.

6. The method according to any one of the preceding claims, wherein the method produces an organoid or an organoid fragment that has a mutational signature.

7. The method according to any one of the preceding claims, wherein the genotoxic bacteria is introduced in the first phase and prevails in the system for further culture in the second phase.

8. The method according to any one of claims 2-7, wherein the method comprises:
(a) the first and the second phase only;
(b) the first and the third phase only;
(c) the second and the third phase only; or
(d) the first, the second phase and the third phase.

9. The method according to any one of the preceding claims, wherein the phases in the method are repeated at least twice, at least three times, at least four times or at least five times, optionally wherein the phases in the method are repeated at least five times.

10. The method according to any one of the preceding claims, wherein in the genotoxic bacteria is in exponential growth phase when it is incorporated into the co-culture.

11. The method according to any one of the preceding claims, wherein the genotoxic bacteria has a multiplicity of infection of between 0.1 and 800, between 10 and 100, between 50 and 100, or about 100 when it is incorporated into the co-culture.

12. The method according to any one of the preceding claims, wherein the growth of the rate of the genotoxic bacteria in the second phase is lower than the growth of the rate of the genotoxic bacteria in the first phase, optionally wherein the growth rate of the genotoxic bacteria in the second phase is reduced by at least 10%, 20%, 30%, 40% or 50% compared to the growth rate of the genotoxic bacteria in the first phase.

13. The method according to any one of the preceding claims, wherein the growth of the rate of the genotoxic bacteria in the second phase is restricted using an antibiotic.

14. The method according to any one of the preceding claims, wherein the organoid fragment or organoid is derived from gastrointestinal tract, stomach, pancreas, lung, mouth, nasopharynx, throat, hypopharynx, larynx, trachea, skin, vaginal, and/or esophagus tissue, optionally wherein the organoid fragment or organoid is derived from the gastrointestinal tract.

15. The method according to any one of the preceding claims, wherein the first phase occurs for between 15 mins to 5 hr.

16. The method according to any one of the preceding claims, wherein the second phase occurs for between 2 days to 3 days

17. The method according to any one of the preceding claims, wherein the third phase occurs for between 1 day to 20 days.

18. The method according to any one of the preceding claims, wherein the genotoxic bacteria is a colibactin-producing bacteria, optionally wherein the colibactin-producing bacteria is from the family Enterobacteriaceae.

19. The method according to claim 18, wherein the colibactin-producing bacteria is pks+E. coli.

20. The method of any one of the preceding claims, wherein the first phase occurs in suspension culture.

21. The method of any one of the preceding claims, wherein the second and/or the third phase comprises culturing in the presence of an extracellular matrix, optionally wherein the extracellular matrix is Matrigel or Basement Membrane Extract.

22. The method according to any one of the preceding claims, wherein the method produces an organoid that has a mutational signature, optionally wherein the method produces an organoid that has a cancer mutational signature.

23. A method for testing the effect of a candidate compound, wherein the method comprises:
exposing the co-culture of genotoxic bacteria and organoids from the method according to any one of the preceding claims to one of a library of candidate compounds;
evaluating the organoids in the co-culture for any effects,
identifying the candidate molecule that causes said effects as a potential drug.

24. The method according to claim 23, wherein the genotoxic bacteria is precultured with one of a library of candidate compounds and then introduced into the co-culture methods of any preceding claim.
